(19) United States
(12) Patent Application Publication
(10) Pub. No.: US 2024/0175021 A1
(43) Pub. Date: May 30, 2024

(54) METHODS FOR MODULATING FMR1 EXPRESSION

(71) Applicants: IONIS PHARMACEUTICALS, INC., Carlsbad, CA (US); THE REGENTS OF THE UNIVERSITY OF MICHIGAN, Ann Arbor, MI (US)

(72) Inventors: Frank RIGO, Carlsbad, CA (US); Peter TODD, Ann Arbor, MI (US); Caitlin RODRIGUEZ, Ann Arbor, MI (US)

(21) Appl. No.: 18/314,691

(22) Filed: May 9, 2023

Related U.S. Application Data
(63) Continuation of application No. 16/982,787, filed on Sep. 21, 2020, now Pat. No. 11,661,601.
(60) Provisional application No. 62/646,803, filed on Mar. 22, 2018.
(60) Provisional application No. 62/740,285, filed on Oct. 2, 2018.

(51) Int. Cl. C12N 15/113

(52) U.S. Cl. CPC C12N 15/113; C12N 2310/11; C12N 2310/315; C12N 2310/321; C12N 2310/3341; C12N 2310/341; C12N 2310/351

(57) ABSTRACT

Provided are methods for increasing the amount or activity of FMR1 RNA, and in certain instances of increasing the amount of FMRP protein, in an animal. Such methods are useful to prevent or ameliorate at least one symptom of a Fragile X-Spectrum disorder. Such Fragile X-Spectrum disorders include FXS, FXTAS, and FXPOI.

### CROSS-REFERENCE TO RELATED APPLICATIONS

This application claims priority under 35 U.S.C. § 119(e) to U.S. Provisional Application Ser. No. 62/646,803 filed on Mar. 22, 2018, and U.S. Provisional Application Ser. No. 62/740,285 filed on Oct. 2, 2018, the entire disclosures of which are incorporated herein by reference.

### STATEMENT OF GOVERNMENT SUPPORT

This invention was made with government support under R01NS086810 and F31NS090883 awarded by National Institute of Health. The government has certain rights in the invention.

### SEQUENCE LISTING

The present application is being filed along with a Sequence Listing in electronic format. The Sequence Listing is provided as a file entitled BIOL0326USL2SEQ_ST25.txt, created on Mar. 19, 2019, which is 128 KB in size. The information in the electronic format of the sequence listing is incorporated herein by reference in its entirety.

### FIELD

Provided are compositions and methods for reducing Repeat Associated Non-AUG (RAN) translation products and increasing FMR1 RNA and FMRP synthesis, in a cell or an animal. Such compositions and methods are useful to prevent or ameliorate at least one symptom or hallmark of a fragile X-spectrum disorder. Such fragile X-spectrum disorders include Fragile X Syndrome (FXS), Fragile X-Associated Tremor/Ataxia Syndrome (FXTAS), and Fragile-X-Associated Premature Ovarian Failure (FXPOI).

### BACKGROUND

Fragile X-Spectrum disorders are caused by varying length CGG repeats at the FMR1 locus, with FXS being caused by repeat expansions containing more than 200 CGG repeats, and FXTAS and FXPOI being caused by shorter, so-called premutation repeats, containing 55-200 CGG repeats. The normal, non-disease associated repeat length is approximately 30 repeats. FMR1 encodes FMRP, an RNA-binding protein that plays a central role in regulating activity-dependent protein translation at synapses by suppressing translation of bound transcripts. Levels of FMRP in FXS can be impaired through two distinct mechanisms: hyper-methylation of cytosine residues leading to heterochromatin formation and transcriptional silencing at the FMR1 locus, or decreased translational efficiency as CGG repeats impair ribosomal scanning through the 5′-UTR. FXTAS patients tend to have unmethylated repeats that enhance FMR1 transcription, leading to neuronal inclusions (Nelson, Orr, and Warren, The unstable repeats-three evolving faces of neurological disease, Neuron, 2013)(Hagerman, Fragile X-associated tremo/ataxia syndrome (FXTAS): pathology and mechanisms, Acta Neuropathol., 2013).

RAN translation from CGG repeats can lead to the formation of homopolymeric polyglycine, polyalanine, and polyarginine proteins. Polyglycine is the most predominant product of RAN translation initiating at CGG repeats and forms intranuclear neuronal inclusions in the brains of FXTAS patients and in the ovaries of FXPOI patients (Todd, et. al., CGG Repeat-associated translation mediates neurodegeneration in the fragile X tremor ataxia syndrome, Neuron, 2013).

Currently there is a lack of acceptable options for treating fragile X-spectrum disorders such as Fragile X Syndrome (FXS), fragile X-associated tremor/ataxia syndrome (FXTAS), and fragile-X-associated premature ovarian failure (FXPOI). It is therefore an object herein to provide compositions and methods for the treatment of such disorders.

### SUMMARY OF THE INVENTION

Provided are compositions and methods for reducing Repeat Associated Non-AUG (RAN) translation products and increasing FMRP synthesis, in a cell or an animal. In certain embodiments, the animal has a fragile X-spectrum disorder. In certain embodiments, such fragile X-spectrum disorders include FXS, FXTAS, and FXPOI. In certain embodiments, compounds useful for reducing RAN translation products and increasing FMRP synthesis are oligomeric compounds or modified oligonucleotides. In certain embodiments, the oligomeric compound comprises a modified oligonucleotide.

Also provided are methods useful for ameliorating at least one symptom or hallmark of a fragile X-spectrum disorder. In certain embodiments, the fragile X-spectrum disorder is FXS, FXTAS, or FXPOI. In certain embodiments symptoms include intellectual disability; physical abnormalities, such as prominent ears; anxiety; depression; ataxia; tremor; memory loss; peripheral neuropathy; occult primary ovarian insufficiency; and polycystic ovarian syndrome. In certain embodiments, amelioration of these symptoms results in increased intellect, lack of physical abnormality, reduced anxiety, reduced depression, improved movement, improved memory, reduced pain or numbness in limbs, improved ovarian function, and normalization of menses. In certain embodiments, hallmarks include neuron death and presence of RAN translation products, such as polyglycine, polyalanine, and polyarginine proteins.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

As used herein, “2′-deoxynucleoside” means a nucleoside comprising a 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil). Unless otherwise indicated, a 2′-deoxynucleoside comprises a deoxyribosyl sugar moiety having the β-D stereochemical configuration.

As used herein, “administering” means providing a pharmaceutical agent to an animal. “Administered prior to the detection of the at least one symptom” is prophylactic administration and means providing the pharmaceutical agent to an animal before a symptom of a fragile X-spectrum disorder is apparent through clinical diagnosis.

As used herein, “animal” means a human or non-human animal.

As used herein, “antisense activity” means any detectable and/or measurable change attributable to the hybridization of an antisense compound to its target nucleic acid. In certain embodiments, antisense activity is an increase in the expression or amount of a target nucleic acid or protein encoded by such target nucleic acid compared to target nucleic acid levels or target protein levels in the absence of the antisense compound.

As used herein, “ameliorate” or “amelioration” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. In certain embodiments, symptoms are neuronal death and presence of RAN translation products, such as polyglycine, polyalanine, and polyarginine proteins.

As used herein, “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include, but unless otherwise specified are not limited to, adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to an oligonucleotide means that the oligonucleotide is complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

As used herein, “increasing the expression or amount” refers to enhanced expression or greater amount relative to the expression or amount in an untreated or control sample.

As used herein, “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” means a modified internucleoside linkage in which one of the non-bridging oxygen atoms of a phosphodiester internucleoside linkage is replaced with a sulfur atom.

As used herein, “MOE” means O-methoxyethyl. “2′-MOE” means a —OCH₂CH₂OCH₃ group at the 2′ position of a furanosyl ring.

As used herein, “fragile X-spectrum disorder” means a condition marked by large (greater than 55) CGG nucleotide repeat expansions in the 5′ UTR of the FMR1 gene on the X chromosome. Fragile X-spectrum disorders include Fragile X Syndrome (FXS), Fragile X-associated Tremor/Ataxia Syndrome (FXTAS), and Fragile X-associated Premature Ovarian Failure (FXPOI). FXS is the leading monogenic cause of autism and intellectual disability. FXTAS is an age-related neurodegenerative disease characterized by gait difficulties, action tremor, and dementia with executive dysfunction and, in some cases, Parkinsonism and peripheral neuropathy. FXPOI is the most common inherited cause of early menopause.

As used herein, “nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases. As used herein, “nucleobase sequence” means the order of contiguous nucleobases (i.e. no additional nucleobases are present between those that are contiguous) in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e. no additional nucleosides are present between those that are linked).

As used herein, “O-methyl” means methoxy. “2′-O-methyl” means a —OCH₃ group at the 2′ position of a furanosyl ring.

As used herein, “oligomeric compound” means a compound comprising an oligonucleotide and optionally one or more additional features, such as a conjugate group or terminal group. An oligomeric compound may be paired with a second oligomeric compound or may be unpaired. A “singled-stranded oligomeric compound” is an unpaired oligomeric compound. A “duplexed oligomeric compound” is an oligomeric compound paired with a second oligomeric compound; this is an “oligomeric duplex.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “Repeat Associated Non-AUG (RAN) translation products” means the synthesis products from translational initiation of a nucleotide repeat expansion in the absence of a normal ORF or AUG start codon. For example, RAN translation of the CGG repeat expansion in the 5′ UTR of FMR1 results in synthesis of polyglycine, polyalanine, and polyarginine proteins.

As used herein, “salts” mean physiologically and pharmaceutically acceptable salts of oligomeric compounds or oligonucleotides (including modified oligonucleotides), i.e., salts that retain the desired biological activity of the parent oligomeric compound or oligonucleotide (including modified oligonucleotides) and do not impart undesired toxicological effects thereto.

As used herein, “standard cell assay” means the assay described in Example 3 and reasonable variations thereof.

As used herein, “sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. Modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid” means a nucleic acid to which an oligomeric compound or oligonucleotide (including a modified oligonucleotide) is designed to hybridize.

As used herein, “therapeutically effective amount” means an amount of a pharmaceutical agent that provides a therapeutic benefit to an animal. For example, a therapeutically effective amount improves a symptom of a disease.

### NUMBERED EMBODIMENTS

The present disclosure provides the following non-limiting numbered embodiments:

Embodiment 1: An oligomeric compound comprising a modified oligonucleotide consisting of 10-30 linked nucleosides and having a nucleobase sequence comprising at least 12, 13, 14, 15, 16, 17, or 18 nucleobases of any of SEQ ID NOS: 11-16.

Embodiment 2: An oligomeric compound comprising a modified oligonucleotide consisting of 10-30 linked nucleosides and having a nucleobase sequence complementary to at least 8, at least 9, at least 10, at least 11, at least 12, at least 13, at least 14, at least 15, at least 16, at least 17, or at least 18 contiguous nucleobases of an equal length portion of nucleobases 3001-3042 of SEQ ID NO: 1.

Embodiment 3: The oligomeric compound of embodiment 1 or embodiment 2, wherein the modified oligonucleotide has a nucleobase sequence that is at least 80%, 85%, 90%, 95%, or 100% complementary to the nucleobase sequence of SEQ ID NO: 1, when measured across the entire nucleobase sequence of the modified oligonucleotide.

Embodiment 4: The oligomeric compound of any of embodiments 1-3, wherein the modified oligonucleotide comprises at least one modified nucleoside.

Embodiment 5: The oligomeric compound of embodiment 4, wherein the modified oligonucleotide comprises at least one modified nucleoside comprising a modified sugar moiety.

Embodiment 6: The oligomeric compound of embodiment 5, wherein the modified oligonucleotide comprises at least one modified nucleoside comprising a bicyclic sugar moiety.

Embodiment 7: The oligomeric compound of embodiment 6, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.

Embodiment 8: The oligomeric compound of any of embodiments 4-7, wherein the modified oligonucleotide comprises at least one modified nucleoside comprising a non-bicyclic modified sugar moiety.

Embodiment 9: The oligomeric compound of embodiment 8, wherein the modified oligonucleotide comprises at least one modified nucleoside comprising a non-bicyclic modified sugar moiety comprising a 2′-MOE modified sugar or 2′-O-methyl modified sugar.

Embodiment 10: The oligomeric compound of any of embodiments 4-9, wherein the modified oligonucleotide comprises at least one modified nucleoside comprising a sugar surrogate.

Embodiment 11: The oligomeric compound of embodiment 10, wherein the modified oligonucleotide comprises at least one modified nucleoside comprising a sugar surrogate selected from morpholino and PNA.

Embodiment 12: The oligomeric compound of any of embodiments 1-11, wherein the modified oligonucleotide is a gapmer.

Embodiment 13: The oligomeric compound of any of embodiments 1-12, wherein the modified oligonucleotide comprises at least one modified internucleoside linkage.

Embodiment 14: The oligomeric compound of embodiment 13, wherein each internucleoside linkage of the modified oligonucleotide is a modified internucleoside linkage.

Embodiment 15: The oligomeric compound of embodiment 13 or 14 wherein the modified internucleoside linkage is a phosphorothioate internucleoside linkage.

Embodiment 16: The oligomeric compound of embodiment 13 or 15 wherein the modified oligonucleotide comprises at least one phosphodiester internucleoside linkage.

Embodiment 17: The oligomeric compound of any of embodiments 13, 15, or 16, wherein each internucleoside linkage is either a phosphodiester internucleoside linkage or a phosphorothioate internucleoside linkage.

Embodiment 18: The oligomeric compound of any of embodiments 1-17, wherein the modified oligonucleotide comprises at least one modified nucleobase.

Embodiment 19: The oligomeric compound of embodiment 18, wherein the modified nucleobase is a 5-methyl cytosine.

Embodiment 20: The oligomeric compound of any of embodiments 1-19, wherein the modified oligonucleotide consists of 12-30, 12-22, 12-20, 14-20, 15-25, 16-18, 16-20, 18-22 or 18-20 linked nucleosides.

Embodiment 21: The oligomeric compound of any of embodiments 1-20, wherein the modified oligonucleotide consists of 16 or 18 linked nucleosides.

Embodiment 22: The oligomeric compound of any of embodiments 1-21 consisting of the modified oligonucleotide.

Embodiment 23: The oligomeric compound of any of embodiments 1-21 comprising a conjugate group comprising a conjugate moiety and a conjugate linker.

Embodiment 24: The oligomeric compound of embodiment 23, wherein the conjugate group comprises a GalNAc cluster comprising 1-3 GalNAc ligands.

Embodiment 25: The oligomeric compound of embodiment 23 or 24, wherein the conjugate linker consists of a single bond.

Embodiment 26: The oligomeric compound of embodiment 24, wherein the conjugate linker is cleavable.

Embodiment 27: The oligomeric compound of embodiment 23, 24, or 26, wherein the conjugate linker comprises 1-3 linker-nucleosides.

Embodiment 28: The oligomeric compound of any of embodiments 23-27, wherein the conjugate group is attached to the modified oligonucleotide at the 5′-end of the modified oligonucleotide.

Embodiment 29: The oligomeric compound of any of embodiments 23-27, wherein the conjugate group is attached to the modified oligonucleotide at the 3′-end of the modified oligonucleotide.

Embodiment 30: The oligomeric compound of any of embodiments 1-29 comprising a terminal group.

Embodiment 31: The oligomeric compound of any of embodiments 1-30 wherein the oligomeric compound is a singled-stranded oligomeric compound.

Embodiment 32: The oligomeric compound of any of embodiments 1-26 or 28-30, wherein the oligomeric compound does not comprise linker-nucleosides.

Embodiment 33: An oligomeric duplex comprising an oligomeric compound of any of embodiments 1-30 or 32.

Embodiment 34: An antisense compound comprising or consisting of an oligomeric compound of any of embodiments 1-32 or an oligomeric duplex of embodiment 33.

Embodiment 35: A chirally enriched population of oligomeric compound of embodiment 1 or embodiment 2, wherein the population is enriched for oligomeric compounds comprising at least one particular phorphorothioate internucleoside linkage having a particular stereochemical configuration.

Embodiment 36: The chirally enriched population of embodiment 35, wherein the population is enriched for oligomeric compounds comprising at least one particular phorphorothioate internucleoside linkage having the (Sp) configuration.

Embodiment 37: The chirally enriched population of embodiment 35, wherein the population is enriched for oligomeric compounds comprising at least one particular phorphorothioate internucleoside linkage having the (Rp) configuration.

Embodiment 38: The chirally enriched population of embodiment 35, wherein the population is enriched for oligomeric compounds having a particular, independently selected stereochemical configuration at each phosphorothioate internucleoside linkage.

Embodiment 39: The chirally enriched population of embodiment 38, wherein the population is enriched for modified oligomeric compounds having the (Sp) configuration at each phosphorothioate internucleoside linkage.

Embodiment 40: The chirally enriched population of embodiment 38, wherein the population is enriched for oligomeric compounds having the (Rp) configuration at each phosphorothioate internucleoside linkage.

Embodiment 41: The chirally enriched population of embodiment 38, wherein the population is enriched for oligomeric compounds having the (Rp) configuration at one particular phosphorothioate internucleoside linkage and the (Sp) configuration at each of the remaining phosphorothioate internucleoside linkages.

Embodiment 42: The chirally enriched population of embodiment 35 or embodiment 38 wherein the population is enriched for oligomeric compounds having at least 3 contiguous phosphorothioate internucleoside linkages in the Sp, Sp, and Rp configurations, in the 5′ to 3′ direction.

Embodiment 43: A chirally enriched population of oligomeric compounds of any of embodiments 1-32, wherein all of the phosphorothioate internucleoside linkages of the modified oligonucleotide are stereorandom.

Embodiment 44: A pharmaceutical composition comprising an oligomeric compound of any of embodiments 1-32 or an oligomeric duplex of embodiment 33 and a pharmaceutically acceptable carrier or diluent.

Embodiment 45: A method of increasing the amount of FMR1 RNA in cells or tissues comprising contacting the cells or tissues with the oligomeric compound of any of embodiments 1-32, the oligomeric duplex of embodiment 33, the antisense compound of embodiment 34, or the pharmaceutical composition of embodiment 35.

Embodiment 46: A method of increasing the amount of FMRP in cells or tissues comprising contacting the cells or tissues with the oligomeric compound of any of embodiments 1-32, the oligomeric duplex of embodiment 33, the antisense compound of embodiment 34, or the pharmaceutical composition of embodiment 35.

Embodiment 47: A method of decreasing the amount of RAN translation products in cells or tissues comprising contacting the cells or tissues with the oligomeric compound of any of embodiments 1-32, the oligomeric duplex of embodiment 33, the antisense compound of embodiment 34, or the pharmaceutical composition of embodiment 44.

Embodiment 48: The method of embodiment 47, wherein the RAN translation product is any of as polyglycine, polyalanine, and polyarginine.

Embodiment 49: A method of preserving neurons in an animal in need thereof comprising administering to the animal the pharmaceutical composition of embodiment 44.

Embodiment 50: A method comprising administering to an animal having a Fragile X-Spectrum disorder the oligomeric compound of any of embodiments 1-32, the oligomeric duplex of embodiment 33, the antisense compound of embodiment 34, or the pharmaceutical composition of embodiment 44; wherein the administering preserves neurons.

Embodiment 51: The method of embodiment 50, wherein the fragile X-spectrum disorder is FXS, FXTAS, or FXPOI.

Embodiment 52: The method of embodiment 50 or embodiment 51, wherein the oligomeric compound, oligomeric duplex, antisense compound, or pharmaceutical composition is administered prior to the detection of the at least one symptom of a fragile X-spectrum disorder.

Embodiment 53: The method of any of embodiments 50-52, wherein the amount of total FMR1 RNA is increased in the animal.

Embodiment 54: The method of any of embodiments 50-53, wherein the amount of total FMRP protein is increased in the animal.

Embodiment 55: The method of any of embodiments 50-54, wherein the amount of RAN translation products is reduced in the animal.

Embodiment 56: The method of embodiment 55, wherein the RAN translation product is any of as polyglycine, polyalanine, and polyarginine.

Embodiment 57: The method of any of embodiments 50-56, wherein the animal is a human.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### A. Certain Modified Nucleosides

Modified nucleosides comprise a modified sugar moiety or a modified nucleobase or both a modified sugar moiety and a modified nucleobase.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the j-D configuration.

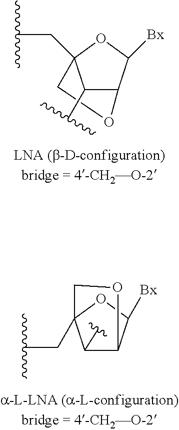

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

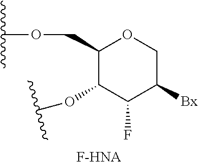

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

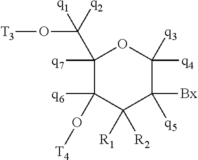

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
   q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q are each H. In certain embodiments, at least one of q₁, q₂, q₃, q₄, q₅, q₆ and q is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

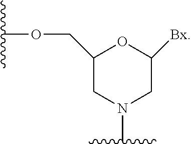

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

### 2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### 3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

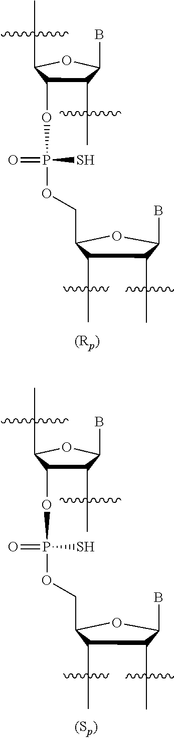

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3-wing (asymmetric gapmer).

In certain embodiments, the wings of a gapmer comprise 1-5 nucleosides. In certain embodiments, each nucleoside of each wing of a gapmer is a modified nucleoside. In certain embodiments, at least one nucleoside of each wing of a gapmer is a modified nucleoside. In certain embodiments, at least two nucleosides of each wing of a gapmer are modified nucleosides. In certain embodiments, at least three nucleosides of each wing of a gapmer are modified nucleosides. In certain embodiments, at least four nucleosides of each wing of a gapmer are modified nucleosides.

In certain embodiments, the gap of a gapmer comprises 7-12 nucleosides. In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

Herein, the lengths (number of nucleosides) of the three regions of a gapmer may be provided using the notation [#of nucleosides in the 5′-wing]−[#of nucleosides in the gap]−[#of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise unmodified deoxynucleosides sugars. Thus, a 5-10-5 MOE gapmer consists of 5 linked MOE modified nucleosides in the 5′-wing, 10 linked deoxynucleosides in the gap, and 5 linked MOE nucleosides in the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target mRNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population.

In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### B. Certain Terminal Groups

In certain embodiments, oligomeric compounds comprise one or more terminal groups. In certain such embodiments, oligomeric compounds comprise a stabilized 5′-phophate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

### V. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

In certain embodiments, the target nucleic acid is a non-coding RNA. In certain such embodiments, the target non-coding RNA is selected from: a long-non-coding RNA, a short non-coding RNA, an intronic RNA molecule, a snoRNA, a scaRNA, a microRNA (including pre-microRNA and mature microRNA), a ribosomal RNA, and promoter directed RNA. In certain embodiments, the target nucleic acid is a nucleic acid other than a mature mRNA. In certain embodiments, the target nucleic acid is a nucleic acid other than a mature mRNA or a microRNA. In certain embodiments, the target nucleic acid is a non-coding RNA other than a microRNA. In certain embodiments, the target nucleic acid is a non-coding RNA other than a microRNA or an intronic region of a pre-mRNA. In certain embodiments, the target nucleic acid is a long non-coding RNA. In certain embodiments, the target nucleic acid is a non-coding RNA associated with splicing of other pre-mRNAs. In certain embodiments, the target nucleic acid is a nuclear-retained non-coding RNA.

In certain embodiments, oligomeric compounds described herein are complementary to a target nucleic acid comprising a single-nucleotide polymorphism (SNP). In certain such embodiments, the oligomeric compound is capable of modulating expression of one allele of the SNP-containing target nucleic acid to a greater or lesser extent than it modulates another allele. In certain embodiments, an oligomeric compound hybridizes to a (SNP)-containing target nucleic acid at the single-nucleotide polymorphism site.

In certain embodiments, oligomeric compounds are at least partially complementary to more than one target nucleic acid. For example, oliogomeric compounds described herein may mimic microRNAs, which typically bind to multiple targets.

### A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligomeric compounds comprise oligonucleotides that are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligomeric compound comprising an oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### B. FMR1

In certain embodiments, oligomeric compounds comprise or consist of any oligonucleotide comprising a region that is complementary to a target nucleic acid, wherein the target nucleic acid is FMR1. In certain embodiments, FMR1 nucleic acid has the sequence set forth in the complement of GENBANK Accession No. NC_000023.11 truncated from 147909001 to 147954000 (SEQ ID NO: 1).

In certain embodiments, contacting a cell with an oligonucleotide complementary to SEQ ID NO: 1 increases the amount of FMR1 RNA. In certain embodiments, contacting a cell with an oligonucleotide complementary to SEQ ID NO: 1 increases the amount of total FMRP. In certain embodiments, contacting a cell with an oligomeric compound complementary to SEQ ID NO: 1 ameliroates one or more symptoms or hallmarks of fragile X-spectrum disorder. In certain embodiments symptoms or hallmarks include intellectual disability; physical abnormalities, such as prominent ears; anxiety; depression; ataxia; tremor; memory loss; peripheral neuropathy; occult primary ovarian insufficiency; polycystic ovarian syndrome; neuron death; reduced levels of FMR1 RNA; reduced levels of FMRP; and presence of RAN translation products, such as polyglycine, polyalanine, and polyarginine proteins. In certain embodiments, amelioration of these symptoms results in increased intellect, lack of physical abnormality, reduced anxiety, reduced depression, improved movement, improved memory, reduced pain or numbness in limbs, improved ovarian function, normalization of menses, neuron preservation, increased levels of FMR1 RNA, increased levels of FMRP, and lack of (or reduced levels of) RAN translation products, such as polyglycine, polyalanine, and polyarginine proteins.

### C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in CNS tissue, including neurons.

### VI. Certain Pharmaceutical Compositions

In certain embodiments, described herein are pharmaceutical compositions comprising one or more oligomeric compounds or a salt thereof. In certain embodiments, the pharmaceutical composition comprises a pharmaceutically acceptable diluent or carrier. In certain embodiments, a pharmaceutical composition comprises a sterile saline solution and one or more oligomeric compound. In certain embodiments, a pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. In certain embodiments, a pharmaceutical composition comprises one or more oligomeric compound and artificial cerebrospinal fluid (aCSF). In certain embodiments, a pharmaceutical composition consists of one or more oligomeric compound and sterile aCSF. In certain embodiments, the sterile aCSF is pharmaceutical grade aCSF.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal, intracerebroventricular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

### Nonlimiting Disclosure and Incorporation by Reference

Each of the literature and patent publications listed herein is incorporated by reference in its entirety.
While certain compounds, compositions and methods described herein have been described with
   specificity in accordance with certain embodiments, the following examples serve only to illustrate the
   compounds described herein and are not intended to limit the same. Each of the references, accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical

modifications. One of skill in the art will readily appreciate that such designation as “RNA” or “DNA” to
describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or R such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, oligomeric compounds and modified oligonucleotides described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: A Reporter System for RAN translation

Repeat-Associated Non-AUG (RAN) translation produces homopolymeric or dipeptide repeat-containing proteins from nucleotide repeat expansions in the absence of a canonical AUG start site. RAN translation can occur in multiple reading frames on the same repeat and occurs in RNA regions typically thought of as non-coding, including 5′-UTRs (Kearse, et. al., “CGG Repeat-Associated Non-AUG Translation Utilizes a Cap-Dependent Scanning Mechanism of Initiation to Produce Toxic Proteins”, Molecular Cell, 2016). In order to elucidate the mechanisms of translation initiation and translation efficiency at RAN translation sites within the 5′-UTR of FMR1, a luciferase reporter system was developed. The reporter system has been described in detail in Kearse, et. al., 2016.

In this system, the gene for nanoluciferase, a fluorescent protein that can be easily measured in cells by conventional methods, is fused to the repeat-containing 5′-UTR of FMR1. Nanoluciferase is fused to the 5′-UTR of FMR1 in different reading frames, as indicated in the table below. Reporter genes were constructed with two versions of the 5′UTR of FMR1, one containing a normal, non-symptomatic repeat length of 25 CGG repeats, and one containing a “pre-mutation” repeat length of 90 CGG repeats, associated with FXTAS and FXPOI.

When nanoluciferase is fused to the 5′-UTR of FMR1 in-frame, (CGG)ₙ FMRP-nLuc, the reporter system “reports” on the expression of full-length Fragile-X-Mental Retardation Protein, FMRP; that is, if a given treatment would increase FMRP expression in a cell, the same treatment will increase nanoluciferase expression in cells containing a (CGG)ₙ FMRP-nLuc reporter. When nanoluciferase is fused to the 5′-UTR of FMR1 with a +1 frame shift, the reporter system “reports” on RAN translation that would lead to expression of polyglycine; that is, if a given treatment would decrease polyglycine in a cell, the same treatment will decrease nanoluciferase expression in cells containing a +1 (CGG)ₙRAN-nLuc reporter.

In a variation of the nanoluciferase reporter system, a green fluorescent protein (GFP) protein was also attached to the 5′-UTR of FMR1 with a +1 frame shift. For this construct, expression of GFP correlates to RAN translation that represents the expression of polyglycine. A different variation of this reporter system has a fluorescent Venus protein attached to the 5′-UTR of FMR1 with a +1 frame shift. For this construct, expression of Venus correlates to RAN translation that represents the expression of polyglycine.

The gene constructs also contain the conventional FLAG tag (Einhauer and Jungbauer, “The FLAG™ peptide, a versatile fusion tag for the purification of recombinant proteins”, Journal of Biochemical and Biophysical Methods, 2001) on the 3′ end. The FLAG tag is commonly used for protein detection by western blot or protein purification with antibodies directed to the FLAG tag.

TABLE 1
Reporter constructs

| | | 5′ UTR | | |
|---|---|---|---|---|
| | | CGG | | SEQ ID |
| Reporter Name | Reporter description | repeats | Report product | NO |
| (CGG)₂₅ FMRP- | 5′-UTR FMR1(25) -exon1 of | 25 | FMRP | 4 |
| nLuc | FMRP-nanoluciferase-3xFLAG | | | |
| (CGG)₉₀ FMRP- | 5′-UTR FMR1(90) -exon1 of | 90 | FMRP | 5 |
| nLuc | FMRP-nanoluciferase-3xFLAG | | | |
| +1 (CGG)₂₅ | 5′-UTR FMR1(25) in +1 frame- | 25 | polyglycine | 6 |
| RAN-nLuc | nanoluciferase- 3xFLAG | | | |
| +1 (CGG)₉₀ | 5′-UTR FMR1(90) in +1 frame- | 90 | polyglycine | 7 |
| RAN-nLuc | nanoluciferase- 3xFLAG | | | |
| +1 (CGG)₉₀ | 5′-UTR FMR1(90) in +1 frame- | 90 | polyglycine, | 8 |
| RAN-nLuc- | nanoluciferase- 3xFLAG-PEST- | | rapidly | |
| 3′UTR | 3′UTR FMR1 | | degrading | |
| | | | protein | |
| AUG-nLUC- | nanoluciferase-PEST | n/a | AUG | 9 |
| Pest | | | translation, | |
| | | | rapidly | |
| | | | degrading | |
| | | | protein | |
| +1 (CGG)₁₀₀ | 5′UTR FMR1(100) in +1 frame- | 100 | polyglycine | 10 |
| RAN-GFP | GFP | | | |
| +1 (CGG)₉₀- | 5′UTR FMR1(90) in +1 frame- | 90 | polyglycine | 18 |
| RAN-Venus | Venus | | | |

### Example 2: Effect of Modified Oligonucleotides on RAN Translation In Vitro

Modified oligonucleotides complementary to a human Fragile X Mental Retardation 1 (FMR1) nucleic acid were tested for their effects on RAN translation products in vitro in cells containing a normal length repeat, with 25 CGG repeats in the 5′UTR of FMR1.

### Experimental Procedure

HEK293 cells were plated at 1.3×10⁴ cells/well and transfected with 100 ng CGG₂₅-FMRP-nLuc RNA or +1(CGG)₂₅-RAN-nLuc RNA using Lipefectamine© 2000 (ThermoFisher) for 7 hours. Media was changed and modified oligonucleotide was transfected at 100 nM with Lipofectamine© RNAiMAX (ThermoFisher) for 17 hours. Cells were lysed in 200 μL of Glo Lysis buffer (Promega), and nanoluciferase levels were detected using a 1:1 addition of prepared NanoGlo reagent (Promega) and analyzed on a 96-well GloMax plate reader (Promega). The levels of nanoluciferase in CGG₂₅-FMRP-nLuc expressing cells correspond to expression of full-length FMRP protein. The levels of nanoluciferase in +1(CGG)₂₅-RAN-nLuc transfected cells correspond to expression of the toxic RAN translation product polyglycine.

### Modified Oligonucleotides

Modified oligonucleotides targeted to near-AUG start sites in the 5′UTR of human FMR1 were synthesized. Each nucleoside of the modified oligonucleotides in the table below comprises a 2′-O-methyl ribosyl sugar moiety and each internucleoside linkage between each nucleoside is a phosphorothioate internucleoside linkage. Compound ID Nos: 1006609-1006614 are complementary to the 5′ UTR of FMR1, GENBANK NC_000023.11 truncated from 147909001 to 147954000 (SEQ ID NO: 1), and 761933 is a control oligonucleotide that is not complementary to FMR1. Each nucleoside of 761933 comprises a 2′-O-methyl group and each internucleoside linkage between each nucleoside is a phosphorothioate internucleoside linkage. The 5′UTR of FMR1 contains three near-AUG sites where RAN translation can be initiated, two of which are in the +1 frame (leading to translation of polyglycine) and one of which is in the 0 frame (leading to translation of FMRP with a N-terminal polyarginine extension, polyarginine-FMRP) (Kearse, 2016). Modified oligonucleotides 1006609-1006614 were designed such that the three most 5′ nucleosides of the modified oligonucleotides overlapped with one of these three start sites, indicated in the “frame” column in the table below.

TABLE 2
Modified oligonucleotides

| | SEQ | SEQ | | | |
|---|---|---|---|---|---|
| | ID | ID | | | SEQ |
| Compound | No: 1 | No: 1 | | | ID |
| ID No. | Start | Stop | Sequence (5′ to 3′) | Frame | NO |
| 1006609 | 2976 | 2993 | CGTCGGCCCGCCGCCCGC | 0 ACG | 11 |
| 1006610 | 2978 | 2993 | CGTCGGCCCGCCGCCC | 0 ACG | 12 |
| 1006611 | 3001 | 3018 | CGTCACCGCCGCCGCCCG | +1 RAN | 13 |
| | | | | ACG | |
| 1006612 | 3003 | 3018 | CGTCACCGCCGCCGCC | +1 RAN | 14 |
| | | | | ACG | |
| 1006613 | 3025 | 3042 | CACGCCCCCTGGCAGCGG | +1 RAN | 15 |
| | | | | GUG | |
| 1006614 | 3027 | 3042 | CACGCCCCCTGGCAGC | +1 RAN | 16 |
| | | | | GUG | |
| 761933 | N/A | N/A | CATTGTTTTTTGTCTTCC | control | 17 |

### Results

Addition of Compound ID No. 1006611 to cells transfected with CGG₂₅-FMRP-nLuc RNA increased nLuc expression relative to untreated control. Percent relative nLuc activity represents the increase of full-length FMRP in cells with a normal-length CGG repeat and indicates that Compound ID No. 1006611 is effective in increasing FMRP synthesis.

TABLE 3
Relative nLuc Activity

| Compound ID No. 1006611 | % Relative nLuc Activity |
|---|---|
| 0 nM | 100.0 |
| 100 nM | 130.7 |

Relative nLuc activity was measured in cells transfected with +1(CGG)₂₅ RAN-nLuc-3′ UTR, representing a normal-length CGG repeat, and 100 nM modified oligonucleotide. Numbers in the table below are normalized to the activity observed with the control oligonucleotide 761933. A reduction in nanoluciferase activity represents a reduction in RAN translation that would result in polyglycine and indicates that Compound ID Nos. 1006011 and 1006013 are effective in reducing RAN translation.

TABLE 4
Relative nLuc Activity

| Compound ID No. | % Relative nLuc Activity |
|---|---|
| None (control) | 88 |
| 761933 (control) | 100 |
| 1006011 | 42 |
| 1006013 | 25 |

### Example 3: Effect of Modified Oligonucleotides on Endogenous FMR1 RNA Expression and FMRP Synthesis Expression In Vitro

Modified oligonucleotides complementary to a human FMR1 nucleic acid at the two start sites for polyglycine (nucleobase 3001 and 3025 of SEQ ID NO: 1) were tested for their effects on FMR1 RNA and FMRP in HEK293 cells.

### Experimental Procedure

Endogenous FMR1 expression in HEK293 cells was analyzed by quantifying RNA after treatment with 1006611 at 100 nM and 250 nM concentrations. HEK293 cells were transfected with a mixture of modified oligonucleotide and Lipofectamine© RNAiMAX. After approximately 24 hours, RNA was isolated from the cells and FMR1 RNA levels were measured by quantitative real-time PCR. The sequence of the forward primer was CATGAAGATTCAATAACAGTTGC (SEQ ID NO: 2) and the reverse primer was CACTTTAGCTAACCACCAACA (SEQ ID: 3). Results are presented in the tables below as normalized FMR1 RNA level, relative to untreated control cells (these conditions describe a “Standard Cell Assay”).

Endogenous FMRP expression in HEK293 cells, which contain fewer than 25 CGG repeats, was analyzed by Western blot after treatment with 1006611 at a 100 nM concentration. HEK293 cells were transfected with a mixture of modified oligonucleotide and Lipofectamine© RNAiMAX. After approximately 24 hours, cells were lysed in RIPA buffer with cOmplete™ protease inhibitors (Sigma). Lysates were heated at 90° C. in SDS buffer for 5 minutes, and resolved on 8% SDS-PAGE gel. FMRP was detected using anti-FMRP antibody ab17722(abcam). Western blots were quantified using ImageJ and normalized with GADPH.

### Results

As shown below, Compound ID No. 1006611 each increased human FMR1 RNA relative to untreated control and indicates that Compound ID No. 1006611 is effective in increasing FMRP synthesis.

TABLE 5
Relative FMR1 RNA

| Compound ID No./ | Relative FMRI RNA |
|---|---|
| Dose 1006611 (nM) | Expression |
| 0 | 100.0 |
| 100 | 110.0 |
| 250 | 216.1 |

As shown below, Compound ID Nos. 1006611 and 1006613 increased the amount of FMRP in a dose dependent manner relative to untreated control as measured by quantified Western blot.

TABLE 6
Relative FMRP Abundance

| Compound | | | | | |
|---|---|---|---|---|---|
| ID No./Dose | Relative FMRP Abundance | | | | |
| (nM) | 0 | 25 | 50 | 75 | 100 |
| 761933 | 100 | 96 | n.d. | 123 | 103 |
| 1006609 | 100 | 100 | n.d. | 101 | 96 |
| 1006610 | 100 | 101 | n.d. | 88 | 102 |
| 1006611 | 100 | 118 | n.d. | 154 | 175 |
| 1006612 | 100 | 149 | n.d. | 127 | 115 |
| 1006613 | 100 | 119 | n.d. | 128 | 140 |
| 1006611 + | 100 | n.d. | 93 | 55 | 58 |
| 1006613 | | | | | |

n.d. means no data.

### Example 4: Effect of Modified Oligonucleotide 1006611 on FMRP Synthesis in Human Fibroblasts

Compound ID No. 1006611 was transfected into wild-type human fibroblasts at 25, 75, and 100 nM for 24 hours and FMRP was analyzed by Western blot as in Example 2. An increase in FMRP synthesis was observed at all tested concentrations relative to untreated control.

TABLE 7
Relative FMRP Expression in Fibroblasts

| Compound ID No./ | |
|---|---|
| Dose 1006611 (nM) | Relative FMRP Abundance |
| 0 | 100 |
| 25 | 138 |
| 75 | 170 |
| 100 | 127 |

### Example 5: Effect of Modified Oligonucleotides on RAN Translation in Cells with Disease-Associated Repeat Lengths

HEK293 cells were plated on a 96 well plate and co-transfected with 100 nM modified oligonucleotide (Compound ID No. 1006611 or Compound ID No. 761833) and 50 μg reporter RNA (+1 (CGG)₉₀-nLuc-3′UTR or AUG-nLuc-PEST) and pGL4.13 (firefly luciferase reporter) using Lipofectamine©2000. AUG-nLuc-PEST is a construct that does not contain any of the FMR1 gene. Instead, it is a positive control for standard AUG-initiated translation of nanoluciferase attached to a PEST signal for more rapid protein degradation. pGL4.13 is a firefly luciferase reporter, which is used as a control for protein expression levels in the HEK293 cells. After 24 hours, levels of expression of nanoluciferase were measured as described above. Levels of expression of firefly luciferase were measured using prepared ONE-Glo reagent in place of NanoGlo and were used to normalize levels of nanoluciferase for total protein expression. The ratio of nLuc:FFLuc in AUG-nLuc-PEST cells treated with modified oligonucleotides reflects the effect of compounds on standard AUG-initiated translation. The ratio of nLuc:FFLuc in +1 (CGG)₉₀-nLuc-3′UTR cells treated with modified oligonucleotides reflects the effect of the compounds on RAN translation in the +1 frame in cells containing a premutation, FTAXS and FXPOI-associated CGG repeat length. RAN translation in the +1 frame leads to polyglycine expression.

No change in the ratio of nLuc:firefly luciferase was observed in AUG-nLuc-PEST control cells or in +1 (CGG)₉₀-nLuc-3′UTR cells treated with control oligonucleotide 761933. The ratio of nLuc:firefly luciferase was significantly reduced in +1 (CGG)₉₀-nLuc-3′UTR cells treated with 1006611 relative to samples treated with control 761933, representing a reduction in the production of polyglycine in these cells.

TABLE 8
Relative expression of nanoLuciferase and Firefly Luciferase

| Reporter DNA | Compound ID No. | % nLuc:FFLuc |
|---|---|---|
| AUG-nLuc-Pest | 761933 | 100 |
| AUG-nLuc-Pest | 1006611 | 81.8 |
| +1 (CGG)₉₀-nLuc-3′UTR | 761933 | 100 |
| +1 (CGG)₉₀-nLuc-3′UTR | 1006611 | 34.6 |

### Example 6: Effect of Modified Oligonucleotides on RAN Translation in Cells with Disease-Associated Repeat Lengths

HEK293 cells were plated on a 24-well plate and transfected with 250 ng+1 (CGG)₁₀₀-nLuc-3′UTR RNA for 3 hours, and then the media was changed and cells were treated with 100 nM Compound ID No. 1006611 or control 761833. Media was changed at 24 hours and cells were lysed in 200 μL RIPA buffer 48 hours after treatment with oligonucleotides. Protein was run on a western blot as described in Example 1 and probed for Flag (mouse anti-flag, Sigma, F1804) and tubulin (mouse anti-tubulin, DSHB, 12G10). Expression of flag-tagged protein in this system represents RAN translation leading to the production of polyglycine. Treatment with 1006611 reduced the production of flag-tagged protein relative to samples treated with control 761933, representing reduced polyglycine.

TABLE 9
Relative Expression of Flag-tagged protein:tubulin

| Compound ID No. | Flag:Tubulin Ratio |
|---|---|
| 761933 | 100 |
| 1006611 | 60.5 |

### Example 7: Effect of Modified Oligonucleotides on Neuronal Death in Cells with Disease-Associated Repeat Lengths

DIV4 primary mixed cortical neurons (rat hippocampus) were transfected with 100 ng mApple and 100 mg+1 (CGG)₁₀₀-RAN-GFP RNA or GFP RNA with Lipofectamine©2000. The mApple RNA was used to normalize GFP signals. Modified oligonucleotides were added immediately after transfection at 1 μM and maintained at this concentration throughout the experiment. Automated longitudinal microscopy was used to track neuronal cell death over 10 days. Neurons treated with +1 (CGG)₁₀₀-RAN-GFP had significantly higher cumulative risk of death than those treated with GFP alone. Treatment of neurons with Compound ID No. 1006611 in combination with +1 (CGG)₁₀₀-RAN-GFP reduces this risk compared to neurons treated with control 761833. Results in the table below are presented relative to treatment with the control oligonucleotide and GFP-only plasmid.

TABLE 10
Relative Neuronal Death in Cortical Neurons

| Compound ID No./ | |
|---|---|
| transfected plasmid | Hazard Ratio |
| 761833/GFP | 1.0 (reference group) |
| 1006611/GFP | 0.88 |
| 761833/(CGG)₁₀₀-RAN-GFP | 2.48 |
| 1006611/(CGG)₁₀₀-RAN-GFP | 2.14 |

### Example 8: Effect of Modified Oligonucleotides in Induced Pluripotent Stem Cells iPSC Cell Line Derivation & Neuronal Differentiation

Induced pluripotent stem cells (iPSC) were derived from a previously established FMR1 patient fibroblast cell line, TC43-97, as well as control normal fibroblasts containing ˜30 CGG repeats. TC43-97 contains a fully unmethylated FMR1 promoter with ˜250-600 CGG repeats. Fibroblasts were cultured in DMEM, 10% FBS, 1×L-glutamax (Fisher), 1 mM MEM non-essential aminoacids (Fisher), at 37° C. and 5% CO₂. For episomal reprogramming, 1×10⁶ fibroblasts were collected after Trypsin treatment and mixed with a set of plasmids pCXLE-hOCT3/shP53, -hSK, -hUL (Addgene), then electroporated with Neon® device (condition: 1650 Volts, 10 mm width, and 3 pulses). Induced fibroblasts were plated onto 6 well plates at density of 0.5-1×10⁴ cells/well and switched 1 day later to a PSC medium mTeSR1 (StemCell Technologies). IPSC colonies appeared and were manually picked and passaged onto new matrigel coated 12-well plates, and continually grown with mTeSR1. IPSCs were passaged weekly using 0.5 mM EDTA and culture medium supplemented with 10 μM Y-27632 ROCK inhibitor (EMD Millipore) for 24 hours. After 5-10 passages the cells were evaluated for pluripotency by immunocytochemistry (ICC) and embryoid body differentiation. FMR1 RNA levels were detected with RT-PCR as described above.

For treatment with modified oligonucleotides, undifferentiated iPSCs were plated as small colonies on MatriGel-coated plates in TeSR-E8 containing 10 μM Rock Inhibitor and grown overnight. Media was replaced with TeSR-E8 the next day. Cells were allowed to recover for at least 4 hours and media was replaced again just prior to treatment. Oligonucleotides (0-100 nM) and RNAiMax (4.5 μl per 100 μl of prepared complexes) were diluted in Opti-MEM reduced serum media, incubated together for 5 min at room temperature, and added to cells. Cells were harvested 24 hours after treatment.

### Results

FMR1 RNA levels were normalized to 18S ribosomal subunit and compared between control iPSC cells and TC43-97 cells. As shown below, TC43-97 cells express less FMR1 RNA as compared to Control iPSC cells.

TABLE 11
Relative FMR1 RNA

| Cell Line | Relative FMR1 RNA level |
|---|---|
| Control iPSC | 100 |
| TC43-97 | 86 |

FMR1 RNA levels were normalized to 18S ribosomal subunit and compared between and TC43-97 cells and in TC43-97 cells treated with 100 nM 1006611. Results are presented relative to control iPSC RNA.

TABLE 12
Relative FMR1 RNA

| Cell Line | Relative FMR1 RNA level |
|---|---|
| TC43-97 | 100 |
| TC43-97 + 100 nM 1006611 | 158 |

FMRP levels, measured by quantitative Western blot as described above and reported relative to GADPH, are reduced in the TC43-97 iPSC cell line compared to the control iPSC cell line.

TABLE 13
Relative FMRP in TC43-97 cells

| Cell Line | % Relative FMRP Abundance |
|---|---|
| Control iPSC | 100 |
| TC43-97 | 11.4 |

Treatment of both control iPSC and TC43-97 iPSC with 1006611 leads to a dose-dependent increase in FMRP, indicating that treatment with a modified oligonucleotide targeted to the upstream+1 near-AUG RAN start site can increase FMRP in both normal human fibroblasts and human fibroblasts containing large FXS-associated CGG repeats.

TABLE 14
Relative FMRP after treatment with 1006611

% Relative FMRP Abundance

| Dose 1006611 | | 0 nM | 50 nM | 75 nM | 100 nM |
|---|---|---|---|---|---|
| Cell | Control iPSC | 100 | 114 | 132 | 151 |
| Line | TC43-97 | 100 | 122 | 150 | 137 |

### Example 9: Effect of Modified Oligonucleotides on Neurons Derived from iPSC Experimental Procedures

Neural induction was performed using a dual-SMAD inhibition protocol, as described in Shi, Y., Kirwan, P. & Livesey, F. J. Directed differentiation of human pluripotent stem cells to cerebral cortex neurons and neural networks. Nature protocols 7, 1836-1846, doi:10.1038/nprot.2012.116 (2012) with some modifications. In brief, two wells of a 6-well plate were grown to approximately 80% confluence, dissociated with EDTA, and plated into a single well of a MatriGel-coated 6-well plate with TeSR-E8 containing 10 μM Rock Inhibitor (Y-27632). The cells were confluent the next day and neural differentiation was induced using neural maintenance media (referred here as 3N) containing 1 μM dorsomorphin and 10 μM SB431542. The cells were cultured for 12-14 days with daily media changes. Neuroepithelial sheets were then combed into large clumps, passaged, and maintained on MatriGel-coated plates in rosette media (3N containing 20 ng/ml FGF) with daily media changes until neural rosettes appeared. Rosettes were manually picked and dissociated into single cells using Accutase. Neural progenitors were plated onto MatriGel-coated plates, grown in neural expansion media (3N containing 20 ng/ml FGF and 20 ng/ml EGF) with media changes every other day, and passaged as needed using Accutase. For differentiation into neurons, neural progenitors were plated at a density of approximately 1.5×10⁵ cells/mL in neural expansion media on PLO-laminin coated plates or coverslips, allowed to grow for 24 hours, and switched to neural maintenance media. Neurons were maintained for up to 6 weeks with half media changes every other day and a full media change supplemented with 1 μg/ml laminin every 10 days.

For iPSC-derived neurons, 150 nM oligonucleotide was diluted in neural maintenance media and added to 6-week old neurons one day after a full media change. Media was changed 24 hours after treatment. Neurons were maintained as above and harvested 6 days after treatment. Western blots were performed as described in Example 2 and normalized to GADPH.

### Results

Treatment of iPSC-derived neurons from control or TC43-87 cell lines with 150 nM 1006611 leads to an increase in FMRP, while treatment of iPSC-derived neurons from the control cell line with the control 761933 does not lead to an increase in FMRP. The upstream+1 near-AUG RAN start site can increase FMRP in both normal human neurons and human neurons containing large FXS-associated CGG repeats.

TABLE 15
Relative FMRP Abundance

| Cell Line | Treatment | Relative FMRP abundance |
|---|---|---|
| Control | Untreated | 100 |
| neurons | 150 nM 761933 | 117 |
| | 150 nM 1006611 | 141 |
| TC43-97 | Untreated | 100 |
| neurons | 150 nM 1006611 | 156 |

### Example 10: Effect of Modified Oligonucleotides on Immunofluorescence Microscopy of iPSC-Derived Neurons

Neurons from the TC43-97 cell line and control fibroblasts were derived as in Example 6. Control fibroblasts contain ˜30 CGG repeats, a length not associated with any disease state. Neurons were treated with 150 nM 1006611 or untreated. Cells were washed 2× in PBS containing 1 mM MgCl₂ and 0.1 mM CaCl₂ (PBS-MC) prior to fixing for 15 minutes with 4% paraformaldehyde/4% sucrose in PBS-MC and permeabilizing for 5 minutes in 0.1% Triton-X in PBS-MC. Cells were then stained for FMRP and the neuronal marker TUJ1 (mouse anti-tuj1, Covance, MMS-435P). FMRP expression is represented as corrected total cell fluorescence (CTCF). The average CTCF is binned for every 5 cells analyzed within a condition, the average of every bin is taken within that condition, and reported as a percentage relative to the Control neuron/0nM group. This is reported in Table 16 below. The average intensity of both TUJ1 and FMRP for control neurons and TC43-97 neurons was also analyzed. TUJ1 intensity reflects the number of neuronal cells, and as can be seen in Table 17 below, was similar across treatment conditions. FMRP average intensity represents the amount of FMRP in the cells. These results suggest that treatment of neurons containing FXS-disease associated CGG repeat lengths with 1006611 can return FMRP levels to those observed in control neurons containing normal, non-disease associated CGG repeat lengths.

TABLE 16
Normalized FMRP fluorescence, average

| | % Normalized FMRP |
|---|---|
| Condition | fluorescence |
| TC43-97 neurons, untreated | 19.2 |
| TC43-97 neurons + 150 nM 1006611 | 55.6 |
| Control neurons | 100.0 |

TABLE 17
TUJ1 and FMRP Average Intensity

| Condition | TUJ1 Average Intensity | FMRP Average Intensity |
|---|---|---|
| TC43-97 neurons, | 504.6 | 199.4 |
| untreated | | |
| TC43-97 neurons + | 496.2 | 429.1 |
| 150 nM 1006611 | | |
| Control neurons | 553.5 | 453.6 |

### Example 11: Effect of Modified Oligonucleotides on Immunofluorescence Microscopy of iPSC-Derived Neurons

Neurons from the TC43-97 cell line were derived as in Example 6. Neurons were treated with 150 nM 1006611 or untreated. Cells were washed 2× in PBS containing 1 mM MgCl₂ and 0.1 mM CaCl₂ (PBS-MC) prior to fixing for 15 minutes with 4% paraformaldehyde/4% sucrose in PBS-MC and permeabilizing for 5 minutes in 0.1% Triton-X in PBS-MC. Cells were then stained for polyglycine. FMR polyglycine expression is represented as corrected total cell fluorescence (CTCF). The average CTCF is binned for every 5 cells analyzed within a condition, the average of every bin is taken within that condition. TC43-97 neurons+150 nM control 761933 condition included 93 cells and TC43-97 neurons+150 nM 1006611 included 194 cells. These results suggest that treatment of neurons containing a long (˜250-600) CGG repeat at the FMR1 locus with a modified oligonucleotide complementary to the +1 RAN ACG site in the 5′UTR reduces the levels of of the RAN translation product polyglycine.

TABLE 18
FMR polyglycine

| Condition | CTCF (%) |
|---|---|
| TC43-97 neurons + | 100.0 |
| 150 nM control 761933 | |
| TC43-97 neurons + | 71.0 |
| 150 nM 1006611 | |

### Example 12: Effect of Modified Oligonucleotides on RAN Translation in Cells with Disease-Associated Repeat Lengths in Neurons

Primary rat hippocampal neurons were plated on a glass bottom, 35 mm MatTek plate and transfected with 2.5 μg+1 (CGG)₉₀RAN-Venus DNA at DIV5. Reporter gene+1 (CGG)₉₀RAN-Venus RNA is described in Example 1. Expression of the fluorescent protein Venus correlates to RAN translation that would lead to the expression of polyglycine in the absence of the Venus reporter protein.

Cells were treated with 1 μM Compound ID No. 1006611 or control 761833 directly following transfection. Cells were imaged after 5 days. Fluorescent signal corresponding to the expression of Venus in each neuron was measured. Approximately 40 cells were counted per each condition, and the control treatment was normalized to 100%. As shown in the table below, compound ID No. 1006611 reduced levels of the RAN translation product polyglycine as compared to the negative control.

TABLE 19
Normalized RAN fluorescence

| Compound ID No./ | Normalized RAN |
|---|---|
| transfected plasmid | fluorescence (%) |
| 761833/Venus | 100 |
| 1006611/Venus | 72.9 |

1-57. (canceled)

58. A method of (i) increasing the amount of FMR1 RNA or FMRP protein in cells or tissues or (ii) decreasing the amount of a RAN translation product in cells or tissues, the method comprising contacting the cells or tissues with an oligomeric compound comprising a modified oligonucleotide consisting of 10-30 linked nucleosides and having a nucleobase sequence complementary to at least 10, at least 11, at least 12, at least 13, at least 14, at least 15, at least 16, at least 17, or at least 18 contiguous nucleobases of an equal length portion of nucleobases 3001-3042 of SEQ ID NO: 1.

59. The method of claim 58, wherein the modified oligonucleotide consists of 12-30, 16-30, 16-20, or 18-20 linked nucleosides.

60. The method of claim 58, wherein the modified oligonucleotide comprises at least one modified nucleoside.

61. The method of claim 60, wherein the at least one modified nucleoside comprises a modified sugar moiety.

62. The method of claim 61, wherein the modified sugar moiety is a bicyclic sugar moiety or a non-bicyclic modified sugar moiety.

63. The method of claim 58, wherein the modified oligonucleotide comprises at least one modified internucleoside linkage.

64. The method of claim 63, wherein the modified internucleoside linkage is a phosphorothioate internucleoside linkage.

65. The method of claim 58, wherein the modified oligonucleotide comprises at least one modified nucleobase.

66. The method of claim 58, wherein the RAN translation product is any of polyglycine, polyalanine, and polyarginine.

67. A method of treating an animal having a Fragile X-spectrum disorder, the method comprising administering to the animal an oligomeric compound comprising a modified oligonucleotide consisting of 10-30 linked nucleosides and having a nucleobase sequence complementary to at least 10, at least 11, at least 12, at least 13, at least 14, at least 15, at least 16, at least 17, or at least 18 contiguous nucleobases of an equal length portion of nucleobases 3001-3042 of SEQ ID NO: 1, wherein the administering preserves neurons.

68. The method of claim 67, wherein the Fragile X-Spectrum disorder is FXS, FXTAS, or FXPOI.

69. The method of claim 67, wherein the oligomeric compound is administered prior to detection of at least one symptom of a Fragile X-Spectrum disorder.

70. The method of claim 67, wherein the amount of total FMR1 RNA is increased in the animal.

71. The method of claim 67, wherein the amount of total FMRP protein is increased in the animal.

72. The method of claim 67, wherein the amount of a RAN translation product is reduced in the animal.

73. The method of claim 72, wherein the RAN translation product is any of polyglycine, polyalanine, and polyarginine.

74. The method of claim 67, wherein the animal is a human.

75. A method of preserving neurons in an animal in need thereof, the method comprising administering to the animal a modified oligonucleotide consisting of 10-30 linked nucleosides and having a nucleobase sequence complementary to at least 10, at least 11, at least 12, at least 13, at least 14, at least 15, at least 16, at least 17, or at least 18 contiguous nucleobases of an equal length portion of nucleobases 3001-3042 of SEQ ID NO: 1.

76. The method of claim 75, wherein the animal has Fragile X-Spectrum disorder.

77. The method of claim 75, wherein the animal is a human.


SEQUENCE LISTING (18 sequences)

SEQ ID NO: 1 - DNA, 45000 bp, Homo sapiens
  source 1..45000: mol_type = genomic DNA
gtcttctgtgctgagaactgtcagagagggtacaacagtggacagtgactgcagggataccagcgaagaagttgctgccataatgcaggtaagagaatggaagcttggactatggttttagctgagcaaatgatcagaaacagagtctggattctacaatgatttgctgatggatctgatgtgtgaaagagggtcaaaatgtctcagttttaggcttgagcaacgaactggaaggcaggagctgcctgtaacgagagacaggagagaaagagagagagagagagaggactttggaagagcaggctcatgatcaggcctgagctcagtgtctgaattcaggtaagctatcttgaaaggggaaatatcaaaagctagagatcagagtaaggctgagactcagagtcaagtggggaagactaagttgcagtatgtactggcagtgaagataagtatttattcattcattgaacataccttgaaatcaaccacttttaatgtgccagggacacaaagatagaaaagacatttgccctgtctggaaggtactaataatccaataaggaaaacagaaatataaataaattattctagtacactaaccatcatagtagaggtattcaacatttgttgagtctctgctatatgccaagcagtgtaatgaggaagcagagggtatgcacaaagttctacaagagcacaaaataagttctggcaaaggtttgtaaagacattcacaagggttttcaccacagtatgacttcagggagttggcagtaacctagatgcccgatcagtagggatatgtatgaataaaatttctggcatactcggtagcaaactaggtgtacacacagcaatgtgggtatagctcaaaaacagactgttgagtaaaacagtgggaaatagagatttacagtccaataccatctctgtaaatgcaagaggcataaacaaaacattatctgtgttaaattatcaaggatctctatcgaacatattgcagcttgtgtctagaagaatgagagtggggatcgagaaagatgaggaaaaaataatataaacactataaaataatgtaaacaaggaccctgtagggactgatatgacaatgtgctgaaaattgaggagcaaagttaactctctgtacctgagataaaataactagctaataggaatccagctgaaaaccttaaggtgcagggcctctatggggcccaggaaggatgtgtagagacatgaacggatgaaagtgcatcacaggttcagggaacaacacaggttgagtgtggcttgtagtaaaaatggttgtgaagagttgacatatttttaagccctgggtaaattgaacaacagcttacacttggaggggtataatcattctaatcaatgtgtccccttttactataatacattggagttgcagctaatgctctgctcccattcagcctatgatgagattctctttcagccctattgggttcttggcctcatgtgactactccaaagaccctagtccaaaaggtctttcctgtttgctatggccttgaggaatgtggccctagatccaccgctttaaagctggagttccaccagcagcaacatcctctcattctggggcacctgcctggggcaggtcatcctgcctctgccaactcagtgctattagttaactctcacctgccatattccagctggaatcatctccccttctccaccccagactaggtcatgttccgccatcatggaagcgcctattcttcataccccttatcacagctgcaactactcatttacttgtctgacaatttgatttatgtccacctactttgctaggtactaagttcaatgctggcagtcgtttcttctttttttttcttttctgttttgctcaccgatttctcgttagcacttagcacagtgtctggcacacgatagatgctccgtcaacttctcagttggataccagcatcccgaagggaacatggattaaggcagctataagcacggtgtaaaaacaggaataagaaaaagttgaggtttgtttcacagtggaatgtaaagggttgcaaggaggtgcatcggcccctgtggacaggacgcatgactgctacacacgtgttcaccccaccctctggcacagggtgcacatacagtaggggcagaaatgaacctcaagtgcttaacacaatttttaaaaaatatatagtcaagtgaaagtatgaaaatgagttgaggaaaggcgagtacgtgggtcaaagctgggtctgaggaaaggctcacattttgagatcccgactcaatccatgtcccttaaagggcacagggtgtctccacagggccgcccaaaatctggtgagagagggcgtagacgcctcaccttctgcctctacgggtcacaaaagcctgggtcaccctggttgccactgttcctagttcaaagtcttcttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggtacttggctctagggcaggccccatcttcgcccttccttccctcccttttcttcttggtgtcggcgggaggcaggcccggggccctcttcccgagcaccgcgcctgggtgccagggcacgctcggcgggatgttgttgggagggaaggactggacttggggcctgttggaagcccctctccgactccgagaggccctagcgcctatcgaaatgagagaccagcgaggagagggttctctttcggcgccgagccccgccggggtgagctggggatgggcgagggccggcggcaggtactagagccgggcgggaagggccgaaatcggcgctaagtgacggcgatggcttattccccctttcctaaacatcatctcccagcgggatccgggcctgtcgtgtgggtagttgtggaggagcggggggcgcttcagccgggccgcctcctgcagcgccaagagggcttcaggtctcctttggcttctcttttccggtctagcattgggacttcggagagctccactgttctgggcgagggctgtgaagaaagagtagtaagaagcggtagtcggcaccaaatcacaatggcaactgatttttagtggcttctctttgtggatttcggaggagattttagatccaaaagtttcaggaagaccctaacatggcccagcagtgcattgaagaagttgatcatcgtgaatattcgcgtccccctttttgttaaacggggtaaattcaggaatgcacatgcttcagcgtctaaaaccattagcagcgctgctacttaaaaattgtgtgtgtgtgtttaagtttccaaagacctaaatatatgccatgaaacttcaggtaattaactgagagtatattattactagggcattttttttttaactgagcgaaaatatttttgtgcccctaagaacttgaccacatttcctttgaatttgtggtgttgcagtggactgaattgttgaggctttatataggcattcatgggtttactgtgctttttaaagttacaccattgcagatcaactaacacctttcagttttaaaaggaagatttacaaatttgatgtagcagtagtgcgtttgttggtatgtaggtgctgtataaattcatctataaattctcatttccttttgaatgtctataacctctttcaataatatcccaccttactacagtattttggcaatagaaggtgcgtgtggaaggaaggctggaaaatagctattagcagtgtccaacacaattcttaaatgtattgtagaatggcttgaatgtttcagacaggacacgtttggctataggaaaataaacaattgactttattctgtgtttaccaattttatgaagacatttggagatcagtatatttcataaatgagtaaagtatgtaaactgttccatactttgagcacaaagataaagccttttgctgtaaaaggaggcaaaaggtaaccccgcgtttatgttcttaacagtctcatgaatatgaaattgtttcagttgactctgcagtcaaaattttaatttcattgattttattgatccataatttcttctggtgagtttgcgtagaatcgttcacggtcctagattagtggttttggtcactagatttctggcactaataactataatacatatacatatatatgtgtgagtaacggctaatggttaggcaagattttgattgacctgtgatataaacttagattggatgccactaaagtttgcttatcacagagggcaagtagcacattatggccttgaagtacttattgttctcttccagcaacttatgatttgctccagtgattttgcttgcacactgactggaatataagaaatgccttctatttttgctattaattccctccttttttgttttgttttgtaacgaagttgtttaacttgaaggtgaatgaagaataggttggttgccccttagttccctgaggagaaatgttaatacttgaacaagtgtgtgtcagacaaattgctgttatgtttatttaattaagtttgatttctaagaaaatctcaaatggtctgcactgatggaagaacagtttctgtaacaaaaaagcttgaaatttttatatgacttataatactgctgtgagttttaaaagtaaagcaaaagtaaactgagttgcttgtccagtgggatggacaggaaagatgtgaaataaaaaccaatgaaaaatgaactgctgtggagaagtgttacatttatggaaaaagaaataggaaccttgttcatcaaattgatagaaaagcttttaaaactaaacaaatcaaacaacttgagtataatggaattcagactttgatttgcctaacataaccaccatatttgcaaggacagctctctatcttctggtgtttattcttaaaaacttaaaagttagatttagcgatcaccagagccactacttttatgcttaggtatttgtttgacttagaaaaaattggtcacgtgtaccactttatagtgccctgcaggtgttaagatatgaaggcactttgacttacacctcataaaatctttacaaagtattttctaaatgaataatgatgaaataaagtctttattctaggtgcatctgccccacataatttgttttctttggactagaagttttgatgtgttgagaatggtaatgaattaactccattttaaatgtagaatgcgtatcactccaatatgaatgccctaatgaatcctaagatttgtaggttttgtgtactagtatgaaaattactaaagatggaaaaatcacatgttggagacataagatacaaacctttttgttttctgaaaatacaacctctgatttctgattccttgttgtaatatggtgtaattatactagattgtaattttgttgttagattatacttttttaagttcagtgtttgaggacagactttcatttggttagtagtattatggcagctagcagctaaatatgataaagtgtacaatcaaaaggatatttttaatgaagatattagtggtctaacatgtcatttcagatacatagctgaaatgtagtaaaatcagttttactacaaataaacttgcataaggtttataaatttataagtttataaatcaacttgggtaaagtgtaaataaacttgcactcgtggtttctctgaagtctcctgagctaactttgcataaaggtgttattctgtacttcgaggaagtgaattattggggtcaaccacattttttttccttcctacagtctgattgccctttttagtttttaggatctttgtggctgcatcatttttccccttttgaactgtgcattttctaaccccatacttaaatattctcataacctccaaattattaattagatgcaacattcagtggtatattactggagtttctgatttctgcccactataggaatgtgcttcctgagaagattgggatcgtgattataataatagttaacaggggatgagtactttctaggtgccaggcactgttctctctgatactttatttgatgtattgttgttattcccattctttaaatgatgcacagagaggttaggtaagtgacttactaccaagtgtcagggccattaagggtcaggattctgaattcctgaaatgatgaaatttagcttgaagaaattggtttgatttcctgcttagttttcaatttcatggtggtctttgattgtattttgtgctataacactgccttagcatcctataactatagttacagtgttatattaccattttttattgttaatacaaagccatcatgaaataattcagtttatgtgccagctttttttgttactaattcttgaaccttggcgctgtacttcttcatgtggatgcctgttaaggaaagataaagttagaaatctttgaccctgctaggaaatttgtctttgttatattgggagctcataaaactgaagtattcaaaagttagaatacatacacacaagaaaaattagtaactaatttaataatgttttctttgcacatgtctctgttgtcttttggtcagagtgaagctaaatgtgtttttcacataatttgtagcctatatgaagtcctggacatgtggtatggttggaaggactgttgatgaggtttattgtctctctttattcttttatgttgttagtgtccccatacaacggggcggggggagtggggacaaaatgataacttgctttatatatgaagccttgggtttgaatcatactgttatcacatttcctatgtccctatcctgtctcctgcaggttttctttcttggttttcttttttggttttctttcttcctttcttcctttcttttcctttctttcctttcttccttttttctttccttttcttttttcctttctttccttcatttctctcctttgctcctttagtttgtttctttgtttctttggtttgtttctttgtttctttggtttgtttctttgtttctttagtttgtttctttgtttctttgtttctttcaacaggtctcactgtcgcccaggttggagtgcagtggtgggatctcagctcactgcaacctccacctctcgggttcaagccagtctcatgcttcagccttccgggtagctgagattacaggcatgtgccaccattcctggctattttttgcatttttagtagagatgaggtttcgccatgttggccaggctgatctcaagttatctgcctgcctcagccctccaaagtgctggaattacaggcatgagacaccgggtccagcttccagcaggttttctttaggagggatattttacaatgctgtaagtttttcctaacgagaattatcatagcactacatgttctgtcttcagtaagtgatacaagcttactgatgatgttgtagttatgttcattggtggtcgggtgtacattgaaactttaacacataatagcctctcctgtgagcagtggttcctgttggtaagatactttactaagggaaggaatgtgaggtgtcgctggggagagtttacccaaataaggatggactttctgtctttgtttcatcagtcctggtaatagaatgtttgaatagatagctctaggcattacatactttcataaatatgattattgtaattacctctttggcccagttgctagtaaattagggaccccttaatgatttatttcctgtttattcaccctgatgaagaacttgtatctcttttaaactgtactttatcgcctttctcaaattccaagattctcatcacattttttttcttcccaaactctaaataaccttttaatattaagtatctttgtggaaacattgttttctttttctatcccaatttttaaagcttttttaaaaaaaagagtgcttttgttgggatgtacattttccaaatgcaaaaacatttatgattctgtgtctcttataaaatatgacactctctacttttctctcatttatttagtgccacctatgtgtgtaatttcattacccacagcagtcttaggaggctggtcgagttccttatttgcagatgaggaatctgaggtccagagatcacttcttggtgagagtctcacagctattaagtattagagccaagattttgaacgtaggtctgattcacagcaaaaccgttaaccactaagtacactgactccagtaagagccctagtcctcacccaatacactttaattcccctgtgcattcattcaaattcattgaatttgctgcctttggaaacctctcaggaacctcctcaacctctcttctctacagacatcagctttgcctgataggtagggatcatagcaaaacacagttttccaaggtggtgataggtggagtgatagtgctctggagatggccaaagaaggaaggtatgagtgtatctgtgggtgggtgagtggtggataaggggaaggacagagccaaaagcgacggctattggaaaaactatgatgagaaacaggaagatggaaccttgttggaattagtgaaagaccttagaattcacaggaggtatttgtccttcacgcgagtgtagaccaaacgtaacctatgagtttcttttattccacttattaaagcagcaaccaaaggtattatataccttctgtattcacttaaaatgactgattttgaaaaagtcatgcaaacatccatttacagataagcctcattaactcaaaggcagtggccctgttgggctctgatgattactcaaaccatttctgacactctgacactacatccggaaatcatccggaaatgcattcagagcctgcaaaagctttttttttttttttttttttttttgaatagctacagcacttgcagatcttcctcctttgagagaatatttgattttaggaaataagcaaatgtagataagtatgattcaactgggcaataacttttaggcaaagagaaaaaaacaaaatatagcaatgtagtaataaggctgatattatagtgcttttatgatgagtctgaaaacagtctccaacatttgtaaatgttttaaatcgggtctgtctactacagtagcccttaagccatatgtagctattgagcacttgacatgttgcaagagtgaattggagactgattttaattttatttaattttaatgtaagtagtcgcacatgacccgtggctactatggtagatggcatagttttagacaagggcagtagtcttgggtagatacttgatttacttagaacatttctttacctagctgtaacaaggttctaatagctgattaaaggacaacatttttagcatgtaatatacagtaaggaactaatgttaattactgccaagatgtataaacattatgaaaccttaaacaaggatgaacacagaagcagtggcccttctttgtaaataaggggtcagttacttcctaataggtgtcttagttttagttaataatctaatagcacccccaaaaagcaaactgttaatttgttattagccatcctgtaaagacaagggagaaatcggggccaggagagtcctctttcccattctccataatttttacgacctatagaaataagagcctaagagtgaccagttcctgcaacctaactcaaaattaggcctctctgttgaagtaatagtgaggcaaacaatcccttgaagcactggggcatgcacccttatgaaatctaataattctaaaatatcattggcagtattaagctagagttctcaaaatatgctgtagaaaatattggtcatttaaatatagctgagtaaaaataagcaagatcaaaatgataagaaaatgttgatttctccccttttgaaccagtaactaactataagggtatatacccatgcttaacttaaaaataattatttagccaccttgggtatagcacaacatatggatgccattatagtccaccttgatcttacaaggaagctttcttttagcgtagctttacttttatttaagcattattgaagaagcttggtatctctgtttaagttgcttctgtatcagtgttttccagggccctgtctcagcttccatagtttttcttaagagcagctaattaatgcttcactccatatgctttaatttgatattttgggaagtttcattttttaaaataatctttttatatcataggcctttgaagacatatttgtttacaaagacatctgcattgaaccatttgtttttaaaaacatgtatcgggcatgttctatataccagaaactatgccatttactgaggacaccgaagttagagaatggtcttaatccgaactagttattgtctcactatccaatatggatatcaaacattcagcgtcatgaggatttactactgtgttttctcccaagagttttataattttagctcttatatttaggtctttgatgcatattcattgatttttttttgtatgcagtatgaggtaaaggttgaaacgtattctttttgcatgtgggtgtccacttgtcccagcttcattttttgaaaacactatactttccccattgaattgtcttggcttctttgttgacaaaggatttattatttctgccacaacccagctgtttctcactgtctaaacctttatgcctgctgccccgtaggcttagtggaatgcctctccatcctccactctcttttgtttagctagtacttccttatcctttaaggggtcagtaggaacctcaccttcttcagtatgttttcctttgtcttatcaacctaccaccatcacccacacacataccttttccctaaggctgggataggaacctcttaggcaatgatctatttgataagtactcactgagttcatactagacactctgcagtatatccgtaaatgaagtaaaacctgacaccaccctcagaaaacttactgttagtgagaaaatgggctataataatagagtggggtaagtttattgtagggataagcataggatgctatgagaatgcagaggacaggaatttaacctagacttctcagttctcccgttgaagttgacatctgaactgaaatctggaagaccagtaagagatagctatgtaaaaagaggggaaggacaataggaaagaaggaatggagagaggcccagaaactacagagtatggcacaagtagtttagcattgttgggtcacaaattctaaggatatgaaagataaagttgaagaagtggtggggctggggaggacagctcctgtagagccttttacagcgggagaaggagtttggctatggtcctgcaggcagggagaaaccactgaaggggagaaacatacagaataaatttgagtaagaagcttaaatctccccaagcccttttaaaataaattaagatataaagcctttgggtttctttatgctttgtcctattcttctaattgtccaaaacaaaacaaaaaccttccttttctgtacctattaaaaggttaattttataaagttacagacagcatgctgattaaagaattcttgataattagctattttgtctgtctttgtgtagattactaacaattgtgtcatcagatttaaaagatctaaacctctggactttatatattttttctacaagtactgtgagattgagaacttaattcaactctagtaactggactttttagtgttgtttgcagcatatgattgttaaagaaagtttagtatatttgtgtgtgcgtatatatatatatatagatagatagatagatttgaggttatatttaatcattgactattggtattctaagattttaaggagagaaaggtaggttacaatatatggctgatatatggttctggaacaattttcttagttttcagtaattctacctaaaatgtacgtccagctttgcactcactaccacattgtaatttccagtatacttgtctatttttcgagatgttgaaatctgtggagaattttttctcatgtttagtgttttagctatgtttatctctagggtatagcacatacaaggtggcaatatagatgtgcttaatgaataaaatgatcttcaaaaactgaccttcatttgaagttctattttattcataaacacataaaacgtttggtatcactgtaaaatttaactaaaaacaaaaactatctttaagctcacaagttaatttaacgttttttcttacacaggcatttgtaaaggatgttcatgaagattcaataacagttgcatttgaaaacaagtaagtgtctcgttatataattttaatgatgaggttctttaatattttatgctaattctactcttcattttttaaaaattcaagtccagtttgagtgcttttcaggaatggatcttcatgttactgactgagaagtttctgaacaactcagtattaaactaatggaatgactgtttctgctaatgtcctggaggtcccttattgtatggtattgatccttacgtcttaattcccttgaatgtgaagaaagaaaccagagagtcttgtgtattagtaactggaagttcgcataggagtatttgtaaattttaaaaagatagtaatgaaaagattcatgggtattgctaaaaagttgtatgtgagttctttaacattaaatctaaaactgatggttttagttgtctagtatattttatggaaacagtcatgtttacctattatctgattttttttaatgtttcatatgttcagcaatagcagtagctgatcttttctgttctcttttgctagatgaagaaataaaaacagtcataggcctaggaatattaactgtatgaaagcatcaatagtatagatgttaacattttattggagaacacaagtctcttgactaaatgttttgaatgctaataaaggctattttcagggtagctgttggtaagattgtaaagtacatataaactccttagtcaaagtgtagatgtggctatgatcttaggattttactaaactctgatggatggttaacagttatcatttttttggctcttatataccaagaaaattaataatatatcaaaagcaggctgcaaatctatagagacagaaagtagattagtgattgcttgtgctggggctggtggggagaacatagctaaagagtacaggatttttgtgtgtgtggcaatgaaaatgttgtaaaattgactggtaattattgcacataactgtaaaccttctataaaccattgaattgtacatttaaattgatgaattataatgatatgtggattatgtctgctaaaatgtagtatgtgacccaggcatactgagcaataaatacacacatcttgcttctaaagtttaaaaccaggcctgtgtttcagtgtctagaaaatatatttcataattgaacttagcagattcttttggctagcaacaggacaaagtagtatccttagatgcctgaaacacgtgtagggggacagagataaaaggattaaaagcatctaaccctatttctggctctggactaataatcgcagaccaaaatacaagtttgaatagtagaatacctttccctgaatacataaagccaaaagttttagtatttgtctcttcgctgtttcatatatccatggctatgcaatagacaacagcatcacaccaacagaggaatctttgataacttggagggtatttttttcttacaattatacactttatctacttacttacatcactaggtaaatgctgtttcccataaagggagagttgtctttatttgctactcttttagtttttcctgtagtctataatctaaagtgttaagcaaacactcatttcctctatgcatttaagaatttgtctagtttatacattgatttattagatcacttctttttaattttcaatcagatttctaccataaacttaaaattatttcatctttggaccttccaattatttatctcctgtaccatatctgcatgaaagtttctcttcaagctctttgtcatcctgggagtttgaaatagatcattgatttttccttgtgttcatggagtatgtttgcaatacaacactgggtttgagcacatcaataggatttaataggtctccaccaaagtaatctctttgcccttccttgtgtgtttcctttttggttttctgttagaaacaaaacgaaaacagtgttttgtaaaacaccagattgtatttaccctttctttcctttcaatcaggaagaagctcctgttactgatagcagattgttttaggtgctttgtttttaggtgcttcctaaaccttccataaagcataaaggtgttttcatatttcacattttttaataaggaatttgaatttttatctgtatacacttaaaatcattattttttccttggaaccattgagttatatattattatatgctttaaaatcaaatttagcaatgaatgtataacatctattaggtgtgtataataactcagaggagggttcagttaatttaggccctaatcagatttccacaaattctgacttaatatttgcccgcttatataacagctcttctttaacaaaaacaagtacttttctcaatagaattttactaagaaagctctttagtaaaacatcgacattatacatacaacatatctcagtatctgctgatgaagaacaccaaaaagaacccagatgtgactgctccggaagttgaatcctcagtatttttgcaaagtttgtctttcagtattttatttgtgtgtgtgtgtgtgtgtgtgtgtgtgtgtgtctatatatatatatatttttttttttttaaagacaggatctcactctgtcacctaggctggagtgcagtggcatgatcatggctcactgtaaccttgaactcctgagcttgagctatcctcccacctcagcctcccgagtagctgggactataggcacataccactgcacctaatttttttttttttttaataatttgttgtaaagatcaggtcttaccttgttgcccaggctgctcttgaagtcctggcctgaagcagtgctcccacctcagcctcccaaagctctgggattataggcttgagccaccgcatcctaatattttatatttttatggatataaaaaataatttggtatctttcagagttgtttaatatcattttaaatttaaaaacataggcaacttaaactcctataggctgtctccatcgggtttctgtggtttaggagaccccaccatcccagtgcatgctgataacgtcatactgatcagcatccagctacccacagcaagaattgaccacctcgtgggatctaaaatttaaagggggaaaagtgagttgtgaattgctaatgtgctgatagccccattttgcttgggaattagagggcagtttttgtggtccttggaatgtggttaaaattcttctgcaagtggaagcatatttatattactaacaattactggtactaatattcaaatattgaaggaaatttctgttgtggacttatgtttaaagctcttagaagttgaaattattggaaagaagacttgttttgaaaatcataatgttgctgtattgtgtttagagaaatattccaaacgggagtaggctgctgtgctgcatgcagactttgctgaaatgttactatattgccgttatgtcccactcagcaaaaactgatgattttaaagcttttgcttcttgaaactagaaaaagaggggtcagccttaaccaaaagttgatggcagagtggtcattatttcagttaaacatgaaaagcatgttaaataattgtatgtttgcttatttacagctggcagcctgataggcagattccatttcatgatgtcagattcccacctcctgtaggttataataaagatataaatgaaagtgatgaagttgaggtgagttttccctgccataaagtcatttagcactgaaagagtggggttaatttatctgtgtttttttttaatactttgtctttaacactgtttaaattactttgagaattacagctggaatggacacgtgcttttgactaactcatcttattaataattcaaaatgatacatgatgcttacatttggctatttgagcagtactcagagcatgtatttgaaagtcactgccgcaagttcctttgcccactagtaattcttttctccttctctcaatttctgaatctttgaatagtatgttgtttgtttacaactgtgtccccattgtaagcaaaatggattatgaaaattaattttacacaggaaagaaatcatgctttattacaaaatagtatactagaatttctttaagtagcagtgaatcttcttggtatatttttaaaaacctacaagctttagtttatacatattggtaaaatctctttttcacaggttataccgtgaactacctgcttatctcccgttgagctctttgaccaaaatgtgctatggagatctaacatgtgaacagaaatgttgtgtttgtgatttctttctccattagaaattggttttgtatttaaggcgcttacagtgcctcatatagtttgtgtggtatcttctctcctttgtatcctttcacatactccattttgagctgcttctcttctttttctacaataataagcataatgccttactattagttaataatagttgccgttccatgaatatttgtagtatgaaggatgtcgctaagtagacatttgtgcaggtgaggcagagtggttctaggtcctgtccttcagttctttatttatttatttatttattttttgagatggagtctcactcttgtcatccaggagtgcaatggcgcgatcttggctccctgcaacctccgcctcccgggttctagcgattcttctgcctcagcctcctgagtagctgggattacagtcgcccaccaccacgcccagataatttttatatttttagtagagacggggtttcaccatgttggtcaggctagtctcgaactcctgacctcaggtgatccactcgcctcggcctcccaaagtgctgggattacagatgtgagccaccatgcccagctggtccttgaattcttatttgctgcactaggtagtaaaggtgtagcaaaaacaggacagaatcttctgcctttgatgaaatcacattccagtaaggatagctaatatgacagatgtgagtcttaaagaaaaaaaaagggagaggatgaaaggtgatttagggacaggattgctgtttcagagagtagtcagggaagatctctcaatgtccaggcctgtgtttttctctctacagttttaaagtggacataggatatagtgttatttattttccaaaaatgtaaccaatctctactaggtgtaagcagctgggcttggttttgcaagggatcttaatagggaacagaaacactgtgtatgcttagctgtccacttccccagaagactgtaagctctctgaaggcaagacccaggtctttggtctgtattaggagtacccagaatagtgtttgatggagggatgaaagggactttttctccccagcttggtcacttgacagatcgggaccaatgagataccattgctaggatgacctcactgtcttacacaccgattcgttgaggtaatttctctggctagagtagttgagaaagatggaagcaagagggaaaggtatgttaagttgtattcagtcactgtattgtgtgatcattaactttggttgtaaaagggagacaagttcgagcttttactctacttgtcagccctagactttgtacctctcttggcctcatgtctttatcccccttgtctttcttatttctcttttctgcttctgagtatccctgtctctctgtccatatatactctgttgtctcttttcctattaaccttgatttgctagggtcttcatgaaaggttccatgctgtacatcttaagtgaactttgaagtggtcatcaaagagatcagttagaagaactgatctgcagtgtgtgacagaccaggaaagtgatatccagaggattgccaccatgaagttgtcatttttagtggagaggaagcaggagaccagaaagtgaccaaggatctgtccatttagttccaggagatatgactgccagagtggtatccttagtgggctgaatgttcagtggtcttactttagtgaccaagcaacaagacagtagggcatggtcagtaactttcagtcaatttttgcttactaggataaagcaaaaataagcttatacatttttagatacattttttgtaacttgctgagtacccaaggaaagtgtgcttgtatttatgggcgtctattttcagagcactaattattgctgaattagaacagaaatataggaaaactgatttttacaaggagcttcaaagcaatctcaggtagtttctgattatgtatctctgcctacctcggggtacatagacagggttacaatttggttgaggatatatgacatgtggtttttaaagacacctaggggcattttaagaaaatttcctcgatatctgaaaatctgtagatttcaaaattatgttaatcatgaaatattctgtgttgtaatttttgtgtaggtgtattccagagcaaatgaaaaagagccttgctgttggtggttagctaaagtgaggatgataaagggtgaggtaggaaaatgcctatttaaatttttttcttatattgtttcctttttttaaacccaggttgtacattcccgtgtggatttctattttgaagtaatatctaattttgagtaatttaattaaaatgttttcactatgtgttcagtatgtttctgttggtcataaattttttcacatagattatttattttaaaataactgaatagggagaacttcttattcttactttaaaaattgtgattagaagtgacttttatttatttctcagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtaagttgatacacaagaaatgctgagaacttggaagtgatatgcaattagtttagaagaatttctagtagtttaaaattttttaaactactagaaattgattttaaatttggtggactttggcaaaaatggtttggtttgcaagagcaatggagaaaaaacttatttaacttttcctacttatagaatcaaggcagccttgcgcttgtttaaacttacttggctatacacgtgtctgatgtacataatgtacatttgttttctaaactaactttgttcattctaagatttaaaaactgatcaaacaaatgtgataattctgtcataagtcacaaactataaacgatttctccgtttttttctcttttacctaaaatgttttcagataaatgtgtaataagtagagccaaagaagctttaaaaagtcatctcatcttacaatccttgatctggaataatgcttaaagactatagaagagacgcatttttattttttacagattttattaagtgaatacagtacgtttgatttggacatgttgttgaacattaaattgcagttcagaatacatagaaatcctcccttttctagagattgtgtatgtgtgtaaatttttgtgccttctggttcatgtacgtttccttctctccctcaaatcgtttagcagtttcttaccccattctccgtgatgtgcttatttcatattgcatgcctgtatcaaaacatctcatatgccccataaatatatatacctactatgtacccacaaaactaaaaattaaaaaaaaaggaaaaaaaaaggcctgcaacaagagaagaagaaagaaatcagataccctgtaggtagctatctccaaaagttttcaatagttgacttaagaaaactactgacttgtatgtaactgaaatcaggaaacctgtagtgtagagtggttcccctgaatatgtctcttttggtaatctaagctgtatttcagaaatgtacagaaaaggagctaagcttctaaatggatatagcatattttgcataaatcaaatttaatatgaatccatgattcttgtatgtgtaaatctgcctgcatttatttatgtcagtagttggtaattattcatcttaatttttttttttaaatttctaggtgtgccaaagaggcggcacataaggattttaaaaaggcagttggtgccttttctgtaacttatgatccagaaaattatcagcttgtcattttggtgagcatttttgagttgtttatttttagtttaattcatctggggcaattgccttgataataatgttgttaatttaaatcatttagtccatcaatgaagtcacctcaaagcgagcacatatgctgattgacatgcactttcggagtctgcgcactaagttgtctctgataatgagaaatgaagaagctagtaagcagctggaggtatgtcactttccctagcactgcttgtaagggtacctaggaacgattaactgtatcattccacaacttgaatatggggagcttgtcatttatttactgcttcttaacaattccttaggttctatagttggaaaattgtacaaagcataatcatactaaagtttccatgcctgaggtttcaaaattaaacagcagcttatgtatattaagggacttctggtactttcacttttactagaagtttatcaaagttgctaataaaatgcggcaatgcttcttccatataacatgttgtcattaaaaatactagagaatatttaaatatctaatcatattcctctcattatgtatgtttatctttgtttaaaagggtatagaacttcttcattctaattggttgtcattctttaaaactcctgtcttcagattcccgccagaggctatttccctaacttactattttgtctcttaaggtgcattttatctacttttattaatcctccaataattttcatcaccatatatttgttttcattgataactatcaagtacgtccatccattactatagaaactaagtggatcttagatgaatgtgccagctctactatattcctgtcaaattccagagttcgtcatcattattcattagaaggaaaacaccacacatttgaaatagtaaagaaaaaaaggagttccaaactgcattttaggagaaaaggtagtcaagactgtgttcttataaaaaccctgtgaaatttgccacctgtcaacttgatgtatgatgctgaactcttagcagaaggggaaaagtttaatctccttttgccactttgttttttcttaaggccatggaggtctgtttaattggtctttgctctcattctttcttgatgtccctgtgttcatgattttgtggttccttcagtgattctctcataacttttagcccagagctgcctccttactttgattgtgagtattatgaaaatatactcatatgcctcttaaacatcgaattattcttaaatgatctatcatacttttggctaaccatttatcagtgaattatcaatgaaaaagaacgaaccagattaatgaaccaggactcaaaaaacatgacttcttgaaaagtcctagaattattttatagattttattactctagtctaaaaaaaatgggtattgcacttttaaattatttatgctagttgtattcattcattcaaattatcaggtctgttgaacaatgagaaactagtatgcatgcctatctttcttcactactaatattaaagagatttacatgttcctataagaaattcccatttttcaacatgagccactaatacgtttggttttcattaatggcatgctagaactgaacactaaattttgataatggtatttaaagacagtggttatccacaagtttgggatgtcagtgtaacttagtttcgtcatcactggatatttacaagtgctcatcataattgtggatccagatcacaaggtcttacggactctggtctcatgttagctttcagatgcttactaagtttttaatttttgacattttttgattattcagtttaactactgtaccaacaatttttgattccttaggttttgagggaggtgtcattttaatgtcatttcaacagaggaatgtttagaccacagtacctaatgatgttataaaaaatggcactgtttccagaaaatatatactgataccttttaataaaaatgaagcgaataaaattttctataaagatgatatttaaacatttttttacctaaataagtaacattgagtttgtcagaccgagtgagctggtttttactggggaacatttgacctgcttcttagttattatattgctgatgtcattggttaactaaggcagccttgtgataagttttcagcagttaccttggaaaatgtcaacaagttctttcctattaacaaaaacatataaaatgtcatagacctttagtaacctaaaaagtacttaggctgtgagaggtattttctaaaacctgttagttttaacctaaaactattaataatttatggacccctcatatacagggtcaagataatttacatggctggcctaaatacagttgtcgtaatagttgataaagtgtattcatcagacgtccatttctcttcagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggtaaatattttactgcatagtttttttttccccaaacaagtatttcagctggctaatcttttgtcttaaaatgtttccccttttattaggatcaggatgcagtgaaaaaagctagaagctttctcgaatttgctgaagatgtaatacaagttccaaggaacttagtaggtaagtcagaagtatctgttgacatatagtacaacaactaagtttaggtaaacagtttatttatagtgatagaattccattttttcttactggagggtatcatttaattgaaacatagtctttgaaatatgatctgttctttttttttattatttaagttttagggtacatgtgcacaatgtgcaggttagttacatatgtatacacgtgacatgctggtgcgctgcacccactaacttgtcatctagcattaggtatatctcccaacgctatccctcccccctccccccaccccacaacagtccccagagtgtgatgttccccttcctgtgtccatgtgttctcattgttcaatcgatctgttcttacagcatacaaaaaaatatttgttaagtatgatgctcaaaggttacattttcagaaggtattcaaaagagattgcttttctagttcaaacctactaatacctgaaaggataaatcttgcccagccctcctgactgtataccttttatatcttaaaattaaataatttttcaaaatgaatgatatttggggactttatgatttgaaatagatttgtatttgatctttcttatgggactataaaatgatagcattcattttaattatctcagggttgaagatctgaacataggttacttaaatctaaaaagttccaaagctagaagaaacttcagatgtctctgggactttctgcaaagtcaatagtaattcttcattttgacttgagtagtgttttatcaccatcaccatcgtgagtatcagcaaatatttaggagctcttcaccaatactcctaaatgctgcagatatctttagtagaacatatcaaaaccaggtgcagttttatcaagaaagctacatatgtttttaatttgtaaataaaattgggacatactgcctatatgttttttatcttttaaacttgagatcatggccattttatataaagtaatacatgttctttggaaactttaaagaagaaagcaaggacttctcatacattctgtttacgtagtttcaatgctaatttagcctgcttttttgtcagcatagactcataaacatcttccttgtcattaacaaacactagaaatgataaatgtatctcccaaagaaaattagcttgaagataaagtgcaaatagtaggaaaataatgcagaagtgttagctcaactgtgtactacagacaactggagaaatgtgtttgtaatgttactaaggtaacttgcataccaattgtatgtgtacagaatgtatgcatagcgcaagataaagtgctaatctcaagacagttgggtgatgaataatatttattttcttctttacagcttgtcaagcaattgcataccttttgtagttagaaaggttattgtaaatttaggaattcttatggaaaaagagctggtagttgttttgttttttttttttaatagcaaatgagtagagaattatgtgtcagattcattaaagttggttttgaagatggcatgatggtagtggggaaagtgcttgggcattggagttggatctggatttcagtcctactttattctttggtggcaatgtgagcgtgggtcactgtcttagcccccctgagaattatttttcttacctatcaaatgtgataataggatccacctcatagagttgatgtgataattaaattggaagatatagtaggggacaattctggacacagtaggggactctgttcttcaccttattcaaaacaactccatttcagtagaacagaatcacagtaaattgctctttgggatttggaaataagtgtaatgtagaattggattatttgctattaaatattaaaagtagattctacctgtggttacctggaaagaagaggatctcagaaataaccagaaagactgctaaaagtaaatcagccttgtttttgttttgttgtttctagttagtaaaatgagataattctgttttagagatggtccactgcaacactgggaacgttggaacttttcagaaagcatttttgtgtaagattctgcaaggcagatctttagagaggtgtcataatcagttttgcatgtgttttgtttgagtcaaccaacgttatttgtgatctctatttttcacaaactctctttggtatgtgcactaatttatcttttcattaaaattggattcttcactcactaagaatgaattggtatgactattcagtcagctatagttaacacacagtacttaggataattaagatattatcttaataaaaacatccccaaatctcatttttcattttgttaactcacttagatctttattaaagtcaacattaattcatgaaaatgtaagtgttgcagtttaaaatcaaactgtgtgaagactccaagtagcttctctcacctcacctttccccttttcgttatagaagaacagaaataattgacagaaggacctagaccaagaatcctgatttaatggtgtcagatctgttctatgaccagtcaaagagtttctaggggtgaatgaggcagccacacacagcaagaatgtcaaagctatgctctgattacaagagccacaagaattttttgtgagaaggcttttcactgagttcttatctagcattggctcaaagcaaatttttgcttccatcttttcccccaactttgccttgagctttgcctttaaaggcgaaaaggaaaaaatggaagatgagatggtgagggcaagcacatggtggatgtggccaatgtaagggtacttaggtaccttgggtgagccctcagaatcagatgtagttcacatgtacatttccagaattagtaattcttaagtttgtaatgtcttgctttaccactcgtgtcatgatgcaaatgaaagcagtcattttccagtaatgtatatgattgtaaaataaataaggaaatgatcaagtacctattttttactggtacttgaaatcccaagttttaatagatgttactgaaaaatacttaaattggtttaaagctttttcattgtggttttgagttatcttaaagtgggttttcttaatcttttactctgtgactaatcccttcactttttaatacattgctaaagtttgtaagtaatgcacaggagtgatactctgttaacacattgaccttgaagaatttgtattccttttggatgtatgtatagctgcttaagccactttcagacttagattatcttttataaatgtgccttccatacagaattctgaaaatatttaccatggacaaagaagcagatttaaatcaggtacagacaagaattgaaagtgttgaataagtgaatatatttcccattctctctacttttttcttaattcttatagaactttgcagaaagaatgatacataccacagtaccagcaagtttaaagcgtacccttttgtgcaaaaataagaggttatctttgaactgccttaaaaatccaaaattaccctatcagtttgcaagtattaatcaactctggtacctgaccaaaggagtttaaacaatgatgtcctaattaaacagaaaaccagattaaccatcttgttgaaaagagtctctactttggttttgaaatcttacaattcaatacatacacatacacataaaaatatctatctatatgaatgtaatagtttacagtagggctgtgcttactgctttgaggtatgtgtttttaaaaccaaacttgatttatttatttcttaggcaaagtaataggaaaaaatggaaagctgattcaggagattgtggacaagtcaggagttgtgagggtgaggattgaggctgaaaatgagaaaaatgttccacaagaagaggtatgttacagtgcgaatattttgtggcacatataataaaagtaaaagttttttatgtgatatgttgaggacctctaatatgtgcataaagtgaatgcaaatattctgattatcaagcatgcctgctgtaattaatgttataattgtagataatggcccttctctgtcctctaatgcagtgagtttggattagtcactttattgaattcttttttcctgatctttaaaatgggtagttataaatttttaaatatgagacatacatcactgggtcgagtgttaatggcaatttaataaaataatatatatgtaaatgcttcgtaaagcactttgtaaatgttaatgttgctgacaaaagcaaaattggaacacaggttttctggctttcatcttttcacttttttagtgtggggattgtgtagtatgtgtgtttattctgtgtgctttaaatttatttctgtgcttaatatctcttccccatcctccttacctttaaattctgtaacccttttatcttcataaatatatgctttttgtagcagcttttaaagtatttgcatggggtagagaggtgaggatgcacctttctgtgcaatagtactcttaagatactgtctcatcaaagagccgtaattttcacatattgtctagctttctttttgaaagattcttttagctaaagttaaatcttttttttctgcgtacaatttgtattcggtaattttataaaccaaaactgtttataataacttacattttttaaaaatatgatgattgataacactaatagagctaaataaagtcttaaattggtcctttttttctcttttgtgttttctgttttttaccaaggaaattatgccaccaaattcccttccttccaataattcaagggttggacctaatgccccagaagaaaaaaaacatttagatataaaggaaaacagcacccatttttctcaacctaacagtacaaaagtccagagggtaagaattacttgtcactttgaattacaatacaagtaatttgtctcagatgtcacaattggtattttggatgttttctctggttagacttgtaggctacttatgttctattttttttccaaatgtaattgccagcttatgagatactacaaattagtgtgatacatacttttcaatttattcacagtaacaactcttatgccataaacacaaaataatggggttgcatttagtggtcacatttttcttttgacagtggactctcttttttccatgccttctctctttaaattttgcaggtatcgtagctcaaaatacaccttctgttagttgcattagttactacttgaaccatattttagaaactaattttctctgtttgaactaatctgtttagaaatgggtttaaaagtcctgcagtgaaagtcctgcgttcaggcttctgtgtatcgtttgttatagttaatgacatcccttgcattccttatactgctttaggtgttagtggcttcatcagttgtagcaggggaatcccagaaacctgaactcaaggcttggcaggtaggaaaacattccttgagaaatacactttcagtttatattttaatgtttattccccttgttaacaaagattacaaatgatcctcaggattagggactggagggaggagtgtttgtgggtatgtggatccattgccctggagttaaagcttaaacaccttcatgccgcttgtaaggcttttcctaattacttgtctagttatcatctttaaattatcttctcccttgcactctagagaaccttaagccatactgaagtactttgagttctctgaacattctcgtgcttattttgcttctagacatatgtacaggtttactctgcctggaatgctttttctcctccacatgtcacctcattttgaccttattccttacaaagctttccttgtgtacttgccgaagatccccacccccttgactggttcaagagccttccccttctgttttgcaatgtgctcacccctagcatagcacttaaacatagatgtttatttgttgatctcctcaaccacattgtaagttctttgagggcagggagagctggtgagaactgtttatggaaaaaaatgttgtggactcatggattaaaagcaaaggtgccattttacaaagaggactataacggcaagtggattggatatgtctcattgccgggcagcccattattccagaacacagagtaacttttttctggggtccgtactaaaggccatcattccattcttgcctttcttcattttaatttactattcaggtctactcgtgaagtgcttaaattagagtggcccttggggtactccagcagtttagacatgttgcaagaaaagatattcgtaaataatttgctccgaagtcaatctaactaaatctgttacataggattttaaagctattttggtgatatttgacccagagggaagggtgtagcattcattccatcacatggtattttgggaaataatacagttaaaggcttttgtaacaaataaagcttgttggagaaaaacccaacatgcagaaatgaattgccagttattaatttgcacaattgtttctaggtttccctataacttgtgacaagaagtagttgttttttttttccctcttaatatatatatatatgggctaggagtacagacatttagcatatgccttttaaaaatctgttcttatgttaagtagtgtccatagagagaagttcaaactttccttgtgattcagccttttaaaattttcagagtacattattgaaatgtgtatgggcttgataaaacttaatgcttgtcaatgtacctatttttttgtctcacagattattcagtgcatttttatcctgatgtttttgtttgtattttgtgtgtactctatttacttggttagcagaaatgatttactttttacttttgtacccttggtatttcattttgattttttttttaagaatttgatcggccaggcgcgatggctcatgcctgtagtcccgacactttgggaggccaaggcaggcagatcgcctgagcccaggaattcgagaccagcctgggcaatgtggcgaaaccccgtctctacagaaagtacaaaaatttagctgggtatgatggcatgcacgtgttgtcccagctatccaggaggctgaggtgggaggaacacctagggcctggggaggtcgaggctacagcgagccattatcatgccactgcactccagcctggataacagaataaaaagaccctgtctcaaaaaaaaaaaaaaaaaaagaggcggaggcgggcggatcacgaggtcaggagatcgagaccatcccagctaaaacagtgaaaccctgtctctactaaaaaaacaaaacattagccgggcgtagtggcgggtgcctgtagtcccagctacttgggaggctgaggcaggagaatggcgtgaacccgggaggtggagcttgcagtgagccgagatcccgccactgcactccagcctgggcgacagagcgagactccgtctcaaaaaaaaaaaaaaaaaaacaaaaaagaaaaaaaaaaaagaaaaaattttgatcaggataaagcctttcataaatgaattagagcatcttatactactcattttgtttatacatctcatggtaaagcttgtataattttgaattatacattttgtgattttgctgatactgaatactagtctttataataatctgatacgtttaaaaggttgctattgatctattcgtattcacagtagatgtgtaaagtagtatatcaactacttttgttttcatatattgatttatatttacagcaaacatttgtatacctgctgttcaccagatcctgtgctagggaattagtcgttatttgcatttttcagattaatctatcattacttttataggatcattgttgcaatttctttttcagggtatggtaccatttgtttttgtgggaacaaaggacagcatcgctaatgccactgttcttttggattatcacctgaactatttaaaggtgagaacagaaagaactttaacttctaatccttttgtactaaaatatacaaactttatagtagatctttcagcagttaggactcattctagcctgtgcaaaacactgtatgacttgtcaaaggtaatagagaaaatacgttgtttggcttataatttttttaaaaatggaagtctttataaatttaagtaccaatataccagttttcaataaaattttatacttccctttattcttctcttaaacccttacactcagtttaggcaatcctgtacatagcctgttaatccatttgatcctttctagcattttggttttttccagacaaaaatctgtgtctataactcttgcctttattgttccttttatgtcatttatcaaggaaatctaagcatttcatgaagttctttgtttaaatgtttaccctatttttctctttactgttattcaaaatacaaactatttgtgtcttctcataaatgttcagtttagttagtgtgatgcggttatgcctccttaaaatttcaaactggaagataggaacgaggaggctacaaggtgtagtggagcaagcactggatgggcaatgcagcaatttacttataagacttcagcaaatgaactagctgtgtgactttaggcaagtcacttccctacactagatgcttcttgaaattccttttggccaaaatgttctatactttaatgaaatggaaactgatctacttcattttcataatgttttatttgtatccatacatatcaaaattataatgtagctggtattaaatgtttttaaaaagcatttcaactctatttatggcattaagaatttttttgctgatttcttgaaagaaataaaaagtacatgatttctgcttgtgtgtaaataagaatgaaaggaagaaaaagtgtaatactgggcatgccattcattcttatggtggccagttaattaaaagtggaaaggtttcatccagcaaggtttacatgggtaggatacctctttgatttttttttaagtatgtttatgtgtaatctctctgtgtgtgtgtctctctcgtaattagcttaaatcatcatgaaggcctggagagttaggattgcattgtatcaaatcccttgttcttttgactaatgttctttcttagatcctttgatttattctcaagaatatcttctttggttatcagcagctgccctgttgccattctttccatcaccagaacttccctcgtttgggcctggcatctctcaccaaaaggtgactgctttgccccagtgggataacctggactactcttctgttttgcaagatattcctattccatgagctattactatacctttggaggaagtcactcactcagctgcagtctctcctttgctcaaattctatgtaaaacagattctggtgtaactatgtaaattgctaatgatggcagagcacagatgaaacggaccaaatccttacctgacctttcttttccagaagctgccacaaccaagattttagaaacttagctttacaggagcttctccaatgactgttcatgtagacagtctctatgataacctctgcttcactgagacgcctactactactatagttatgaatctgattccccgtagccagtaaatgagatgtttatgagtggattggaaggacagtggttagaaatgaggtttcatctggttttaacagaacccaacatactggaagtgctttttgtaagggtctgtcattggaccccaggtgtatatttctatcaactaaatgtatactgtttctcgtacttgttgtagcagagttgtagtctttaggaagttttcaaggcatgatgtaagacctagctgtctggtcagctattaaatagttttgttttcccctcatgaggaatagtcagcattttcagttgatgaaaaaatcattgttccttaaccatagtatatgttcttttaaaacaaaagatggcccaaactcaatgctttgtaaattaagatattaactggcaaatctattttaatcaagattatttacaatgtgtgatttttaaggttagctggttataccttgaatgtcatttatctatattcatacattttagcatgttttatttaatatcctatcatgaagaagtccccaaaatagcatatctgtgccttagcagtagaataattggataatagtgccaaattagacaactttaaattttattcagtataattttactgctgagtgtttttgttatatctctaattcttgacatcaataatctgttatctgtagtaattttctttttaaggagatcattcaatttcctgataattctgtgtaacagtgtttactgtaaatttagaactgaaattgaaatattccagtatatttttatctgatgaaaaggagaaaggttttattaagtaaaatgtcaaattatttttactgttatcttgtatattttaaataggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggtacaaactaagcattttactcagtaactttatctgttcctagacttatagctgctaatctctaatattcattagaaccccattataacaatttgccgctacatggtttccaattcacagtgggttaaattgtgttacaggagacatagagtaaaaacctgatgataaacacgtagagttcacagggccggtgcaagttctgttcatgctctgattgggggtaaatctgcaaagcccttgtgtgttgatgattttcttaaggcctctcctgattttataactgtatagtcaaaacttttttcatttgtgtactgtggctcatttttattttgtcaggtatgtgctggtcatacttgctggatggactgtgtaaatcaggggtctgcctcttgtggctcataaggtgattttgagtgacatggccaaagtcagccctaggcacagtcaagaagtaggcgaagtttctctttgccttcccagcttttctgtccgtgcttctttcttctctcccagtacctttcagtgaacctgatccccacctgccagtgcctactgtctttcttgctcaacagtgtatctcctttgtaacttgctaatgatggtataaggtataatccatttcacgcatatttgcatttcaaaaaggaactctttaagatggaaaagccttcaagatccacaggaatgaagctataatggtgaagctataattggtcattagctccaacagaggaagagagaggtagtagagctgatattctggagttggggagagttacctactcctaaggccaaccttgaacctaacccctaacccctctcccaacagtctgttctttttactcatgaatttctgcctcaccgtggagctctttttctccgctcctgtcctctaagtcgttaaccccttcaggcataatgaatttattatagtaattctttattaatgatcaattattgtaatgggagtgggagggcaggttgtggaccaaacatcaggcaagcatgtatctgccttcagctagatccaatccatgacccacaagagacccttccctgaagcagttgccatggtggcctcctttgccaccctctcagaaatgaccaaaatatatacattcccctctgtcctgggccaaatcttgctaaaaggagctctctttttagaggttttgtttatcagggtatcacacttaactgtcgtggataatcttttcctccagagagtatagatgtttaaaggaacacagctatttaatgatatggcacatcaaggtttgaacttaggtggctaataacatacctttttaaaaatgagaatgaaacatgtttataaccaaaattaacctcaaatattgcaaagcccttcattttgagtttaaaattttgttttatgaaatggaaagtgataatactctttgtgggcttataaattgggaagagttttggattacccctgaaacgtctctggaagcttctgttaaccctcttttttttttttttaaagtcagacaatggtatataacttttaactctcgataggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggtatggagatcttcattaagaaatcaaagtgaattgtaacagctgtcttgaagttccatgagaaatcctattgatgcaatgaactgttaccaagatcccatctctcccgttttgtgctgataccataggaaaggatcagccttccacttgtgtagaaagaaagaatattaggcagccttccttatggttcatacagatatatgaataaatgctgataactaattcagcatcttggtgggtctaaacagcatcccttcattaactcacattgacctgtagatgcagaattataataactgtgaaatattttctgttacttagcagcaggctagattattagccattgtgtaactggaatcactggggtattgattacatttcagattctgtggttggtttaaatagagggtgtgggaataaagaacttccagtaagcatttcagaatcagtaactgttgaaccttttgaaaatattctcataggaaacgacgatcactcccgaacagataatcgtccacgtaatccaagagaggctaaaggaagaacaacagatggatcccttcaggtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaatatgcagtctgagtatttgtgaagaccttgacagccacactatgattttattatttattatttatttttatttttttgagacgggagtttcgcttttgttgcccaggctggagtgcaatggcgcaatctcggctcagagcaacctctgcctcccgggttcaagtgattctcctgcctcagcctcctgagtagctggaattacaggcatccactaccatgcccagctaatttttgtatttttagtagagacggggtttcaccatgttggtcaggctggtcttgaactcccaacctcaggtgatctgcccaccttggcctcccagtgtgctgggattacaggcgtgagcactgtgcccagccacactatgattttaataccacattggtagacctgcaatcccccagagaaggaacatcctgggatgtacttagagacctcagtaaaccttatgtgtctttgctcccccaaattaacagggatagggtattctctttgaatagaaaataaggaaaagaaggtgtctgtcttacaactaataatcatgatagacagagctgatgatattcagctagtctccatgcttggtcagtcatctgcttatttacagccaggacctagtccgaggatctcttctcaattggctgtcaccagtgctaaaccaattgttaaataattttaaaattatctcctgagcacatccaggcacctttgctaagtgctttgtgtatgttttatctcatttaatcttacagcaacccactgaggtagttttattattccactttataggtgagtgcactgtagcttcagtcctttgcccacagtcacatagctagcatatgtccaaatgatagtcaaaccttagtgtctgattctctgaatctatgttgtcgtctactttttctgtaacttttaattattaagtagttagggttccttcctgactatggattcatttgggaccatcacccaagattagtgagagatactttttaagacaaagtttatgatggaatatttcttggaattcatagcagctcaaagtaagtgttaactattggtgggttcttaaggaccagcatgtccaagggatagatagattggtaaattactgtatgtacctgtcttttacaaagctataagatatttaagtacccgttttgtaattttccacttaaactgatatgtttgatgaacttggcagtctaactgcattggttaaaaggatgcacatttatatttctaaatgaaatggaatactgaatttaaaaggtgatgttttctctattaagtatgaaaatagatacaaatctggccaggcgcggtgctcacgcctgtagtcccagcgctttgggaggccaaggtggtcagatcacctgaggtcaggagttcgagaccagcctggctaacatggtgaaaccccatttctactaaaaatacaaaaaattagccagacgtgttggcgggcacctgtaatctcagctactcgggaggctgaggcaggagaatcgcttgaacccaggtggcagaggttgcagtgagccaagatcttgccattgcactccagcctgagcaacaagagcaaaacaccatctcaaaaaaaaaaaaaaagggccaggcacggtggcgcacgcctgtaatcccagcactttgggaggccaaggcgggtggatcacaaggtcaggagatcgagaccatcctggctaacatggtgaaaccccatctctactaaaaatacaaaaaattagccgggcatggtggcgggcacctatagtcccagctactcaggaggctgaggcaggaggatggcgtgaacctgggaggcggagcttgcagtgagccaagatcgcgccactgcactccagcctgggcgacagagcgagactgtctgggaaaaaaaaaaaaaaagatacaaatcaaagtactgaatccttggtaacgagacatttaaaacacatgcacatacccactacttaaacatactttgaaattacaaccatttggggatgtttttagcatttgtgcttgaagtagatcaatatttgtagttgttttagttccatttgtcactgttaactttcatttgtacctctggaattagcagtgctgtattcagcattggcacttaaaatattttatagctcttagaacactattttttaaattacattgatctttatactctcctaggattccagtccttccacaaattattatatgcagttttctttatactgttccattcttcagacagagttcaggttgtaggatcatctgaattttcttagtgaaatacaagccagtagaaaccaaagctcgtgtttttctactgtatgactatgggagctctcagtatagagaaacttgatcatacctgtgatagatataaagatgtaaattaaagtctagttagtgagttatttgagggtaaagccagaataggaaaacattttgtttctcttttcctgggcttcacaatctttggagaataactaattttattttaactctagtgttagaggcatgtatgtcttctggtaatcagtaatagtaattcattttacctgtttgtttctcactgttttattctacttagaagaagacatgataggattgtgagtttttctctaactttggatgcagtgagatgaccagtgtgttccagttaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaaaaagttgcacttatgaaaaagcaaaaaattagtctgacagatgtttgctcctggttttaaatttctacatttgacaaaaactaatgatatgtggggggaaagttgtgcaaagtaaatgtatgttgcaaataatcttttatgagcccttaaaaggcaaaaacgaagacacttgaaaataattctccatattcccagacataaaatttcaacacatttattacatagggtgccactaaaccatattaaacatatactcctatttgtgcacacatgaacataccctgccctttggaaaaaagtatgtctatgaagtaaaaaaaaaaaattgatttaaacgtctcagtaataaaataaatgagaggtttgtaggtagaatgtgcctgggaattgactactgtaatcacttcaaggaactctggcacagcctacccgcctaaaaggtacaaatggagaggtgacagtgtcagtgttctacccagggttagaatccaaaatgatgctagacagcctcctctgcattgatgagtttttttccccagaaaaataactattttctgcttttcttgtttttcattctgatgcttttgtggttctttgctacccattctgtctaactgggcatcattagtggtgggtaggctgaaaccatactcaagtaacttgtttggattttccttgtttcacagtagttttcagcactacacatgaacagcctgttggaggcagccagcttaacactcaacttggctctctcagagccatctctctcatctgatgatggtcttagaattcctggtggctcggaatatacgtcttttagaggcagcaaaatgattctgacccttttgagccagtttcagtttcatggccataaacagccttgtgaacctctatgcctctgataaccagttattctgaaatagtcattaccatgtgctagtcttgattgttcataaggcttaatgtaccagaatttaaatcttgagcatcttgttttctctgtttatctgaggtcatttaaaaagttttatttcaatttctctctatgtaccataatgaaactgggaaaatccattttattttaattttttttagtatactaaggggttttctgttagtgagagaggcttgctctgtgttaaactcacagggaagaattatccttcccctggaatacagacccagggtgcagctagcagtgctctggcctttgcatgagagaataatatccatgtgtctcttcctgaccttttgctgacaagtttccctcattaaaaccactttcttctgtgtggctttgtgcagtttgccataacccattacctcaaaaatggtgcccaacatgaggtaaaatccgcttatttcacctctggctttttggaaactgcaaaagggatcaactaagctatactagggatggacagcatccctgctggggctggtgatgggaaacccagccaagctaacatctgcaaagcatgctagtgtggtgcttattggtgttcatgtgccacagaggtcctgggaagtttgtgtaagggagattgagacaattaaatcctttggcactccaagtctggcagcctctgtgagacctaaggaaaaagaagttgggagaaaaccctcagcacctaagtgccattagaatagttgatgcagtctaaacgtttttttgtttgttttggtttttttttttttttgagacagggtctctgtcacccaggctggagtgcagtggcatgatctcagctcactgtggcctccgcctcccaggttcaagcgattcctgtgcctcagcctccctagtagctgggactacaggcacctgtcaccatgcccaactaatttttgtatttttttgtagagatgcagtttcgccatgttggccaggctggtcttaaactcctaacctcaggtgatctgtctgccttggcctcccaaagtgctgggattacaggcctgagccactgcacacagccacaacttttagatttgagacttgtcattgtgcagtgtgccacccaggggcctttggtaggaattgaggtatatcctgtgggatttcaaagacactaacagggaattggactcccagcaatggtcacaagagtaacctgaggctttaattgtttatatgatgacgaggctacacctacaaagtagtgaagctgaatggcaagcactttgggtttatcacgggattctcttctgccagatgaatttgctaggctgaccaatggggagggagcaaatggacctgccttgttcttgaattggatgttcagggcaatcagattcacataaccaccaagaggagacttcctgagaaactgagtgccaagtgaaccattctactgaggttaaagctgttttgagacaacttggcatttttgcatacttgcatgaaaatcctaggcatctaggataagtagtaaccaaatgaccttaattcagagaatgctggaaaagttgatggtttcttatacggagacatccctaactttgctccttctgccagagtgtaaaggatgaaataatcctttggagctgatatatgccccacccaaagtgggggtaatgagataaggggccctggaaagaatatgtctcagcaaaaatgaaaagtcaagggaagagcatcaactctagacttttcctaggcttgcacaggcagtacatcatgactcttggtctgcaacaaagtgatgactagctaaggagatagataggaaaggtagaatcactctaaggagcttgcagagagaaggatgcaggaaacaacactgatattagagtaggcattaagaacccccctatggagagctgatgcaaggaggttctggatgggagggttccctttggagatggaggcagttagctttccccactgcttggccagcaaa

SEQ ID NO: 2 - DNA, 23 bp, Synthetic construct
  source 1..23: mol_type = other DNA
catgaagattcaataacagttgc

SEQ ID NO: 3 - DNA, 21 bp, Synthetic construct
  source 1..21: mol_type = other DNA
cactttagctaaccaccaaca

SEQ ID NO: 4 - DNA, 6328 bp, Synthetic construct
  source 1..6328: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgttaacagatctgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggaggcggcggcggcggcggcggcggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgaaagcggccgcggggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggactacaaagacgatgacggtgattataaagatcatgacatcgattacaaggatgacgatgacaagcgatcgtaatctagaggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 6523 bp, Synthetic construct
  source 1..6523: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgttaacagatctgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggaggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgaaagcggccgcggggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggactacaaagacgatgacggtgattataaagatcatgacatcgattacaaggatgacgatgacaagcgatcgtaatctagaggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 6263 bp, Synthetic construct
  source 1..6263: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgttaacagatctgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggaggcggcggcggcggcggcggcggcggcggcggcggcggcggcggcggcggctgggcctcgaggatatcaagatctggcctcggcggccaagcttggcaatccggtactgttggtaaagccaccggggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggactacaaagaccatgacggtgattataaagatcatgacatcgattacaaggatgacgatgacaagcgatcgtaatctagaggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - DNA, 6460 bp, Synthetic construct
  source 1..6460: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgttaacagatctgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggaggcggcggcggcggcggcggcggcggcggcggcggcggcggcggcggcggcggcggggcggcggcggcggcggcggcggcggcggcggcggcggcggcggcggcggcggcggcggcggggcggcggcggcggcggcggcggggcggcggcggcggcggcggcggcggcggcggcggcggcggcggcggcggcggggcggcggcggcggcggcggcggcggcggcggcggcggcggcggcggcggcggcggctgggcctcgaggatatcaagatctggcctcggcggccaagcttggcaatccggtactgttggtaaagccaccggggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggactacaaagaccatgacggtgattataaagatcatgacatcgattacaaggatgacgatgacaagcgatcgtaatctagaggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - DNA, 7707 bp, Synthetic construct
  source 1..7707: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgttaacagatctgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggaggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggctgggcctcgaggatatcaagatctggcctcggcggccaagcttggcaatccggtactgttggtaaagccaccggggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggactacaaagacgatgacggtgattataaagatcatgacatcgattacaaggatgacgatgacaagcgatcgaattctcacggctttccgcctgaggttgaagagcaagccgccggtacattgcctatgtcctgcgcacaagaaagcggtatggaccggcacccagccgcttgtgcttcagctcgcatcaacgtcttaatctagaactgcataatctagaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 5977 bp, Synthetic construct
  source 1..5977: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggcaatccggtactgttggtaaataagccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggactacaaagaccatgacggtgattataaagatcatgacatcgattacaaggatgacgatgacaagtaaggccgcgactctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - DNA, 6487 bp, Synthetic construct
  source 1..6487: mol_type = other DNA
atcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgcacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccagcgaatctcgggtacgtgttccggacatggggctcttctccggtagcggcggagcttctacatccagccctgctcccatcctcccactcatggtcctcggcagctccttgctcctaacagtggaggccagacttaggcacagcacgatgcccaccaccaccagtgtgcccacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtaccgagctcggatccactagtccagtgtggtggaattcgttaacagatctgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggaggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgaaagcggccgcaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagggcccaaccggtatctagaagatctagagtcgacctgcaggatatcgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgtaccgagctcgcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgattgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaataagcggccgcggccatgccggccactagtctcgagttattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgaagacgtcgcctcctcactacttctggaatagctcagaggccgaggcggcctcggcctctgcataaataaaaaaaattagtcagcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccatctgcagtgaataataaaatgtgtgtttgtccgaaatacgcgtttgagatttctgtcccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagttgatttttgggcatacgcgatatctggcgatacgcttatatcgtttacgggggatggcgatagacgcctttggtgacttgggcgattctgtgtgtcgcaaat

SEQ ID NO: 11 - DNA, 18 bp, Synthetic construct
  source 1..18: mol_type = other DNA
cgtcggcccgccgcccgc

SEQ ID NO: 12 - DNA, 16 bp, Synthetic construct
  source 1..16: mol_type = other DNA
cgtcggcccgccgccc

SEQ ID NO: 13 - DNA, 18 bp, Synthetic construct
  source 1..18: mol_type = other DNA
cgtcaccgccgccgcccg

SEQ ID NO: 14 - DNA, 16 bp, Synthetic construct
  source 1..16: mol_type = other DNA
cgtcaccgccgccgcc

SEQ ID NO: 15 - DNA, 18 bp, Synthetic construct
  source 1..18: mol_type = other DNA
cacgccccctggcagcgg

SEQ ID NO: 16 - DNA, 16 bp, Synthetic construct
  source 1..16: mol_type = other DNA
cacgccccctggcagc

SEQ ID NO: 17 - DNA, 18 bp, Synthetic construct
  source 1..18: mol_type = other DNA
cattgttttttgtcttcc

SEQ ID NO: 18 - DNA, 6678 bp, Synthetic construct
  source 1..6678: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgttaacagatctgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggaggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggctgggcctcgagcgcccgcagcccacctctcgggggcgggctcccggcgctagcagggctgaagagaagatggaggagctggtggtggaagtgcggggctccaccgcggagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagccctgcaggaactgcataattctgaagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc